(19) United States
(12) Patent Application Publication
(10) Pub. No.: US 2024/0400476 A1
(43) Pub. Date: Dec. 5, 2024

(54) RUTHENIUM PHOSPHINIMIDE COMPLEXES AS ACTIVE OLEFIN METATHESIS CATALYSTS

(71) Applicant: Kansas State University Research Foundation, Manhattan, KS (US)

(72) Inventor: Peter Sues, Manhattan, KS (US)

(21) Appl. No.: 18/697,081

(22) PCT Filed: Sep. 30, 2022
(86) PCT No.: PCT/US2022/045418

§ 371 Date: Mar. 29, 2024

Related U.S. Application Data
(60) Provisional application No. 63/270,110, filed on Oct. 21, 2021.
(60) Provisional application No. 63/251,218, filed on Oct. 1, 2021.

(51) Int. Cl. C07C 6/04; B01J 31/18; B01J 31/22; B01J 31/24; C07F 15/00

(52) U.S. Cl. CPC C07C 6/04; B01J 31/1805; B01J 31/2286; B01J 31/2404; C07F 15/0046; B01J 2231/543; B01J 2531/821

(57) ABSTRACT

Catalyst materials for olefin metathesis reactions. The catalyst materials comprise a ruthenium phosphinimine complex. The ruthenium phosphinimine may be synthesized without bound N-heterocyclic carbene (NHC) or phosphine ligands, thereby providing significant advantageous in conversion, selectivity, and stability compared to existing ruthenium-based catalysts.

### CROSS-REFERENCE TO RELATED APPLICATIONS

The present application claims the priority benefit of U.S. Provisional Patent Application Ser. No. 63/251,218, filed Oct. 1, 2021, entitled RUTHENIUM PHOSPHINIMIDE COMPLEXES AS ACTIVE OLEFIN METATHESIS CATALYSTS, and U.S. Provisional Patent Application Ser. No. 63/270,110, filed Oct. 21, 2021, entitled RUTHENIUM PHOSPHINIMIDE COMPLEXES AS ACTIVE OLEFIN METATHESIS CATALYSTS, each incorporated herein in their entireties.

### BACKGROUND OF THE INVENTION

### Field of the Invention

The present invention generally relates to catalyst materials for olefin metathesis reactions. In particular, the present invention relates to ruthenium phospinimine complexes that can be used as catalyst materials for olefin metathesis reactions, as well as the synthesis and use of such materials.

### Description of Related Art

Olefin metathesis is a versatile reaction that can be used to generate a variety of products including pharmaceutical agents and plastics. There are a wide range of pharmaceuticals that use olefin metathesis catalysts in their production. There are also a variety of plastics that can made with these catalysts.

The ability to build increased molecular complexity is critical for academia, as well as for many chemical industries. Therefore, methods that facilitate the selective formation of C—C bonds are highly valuable. In this context, olefin metathesis (OM) is a powerful synthetic tool. One of the reasons that OM is so useful is because of its versatility; there are multiple ways to implement this transformation using the same carbene catalysts. Ring-closing metathesis (RCM) is widely used in natural product syntheses, and cross metathesis (CM) is a popular organic methodology. Ring-opening metathesis polymerization (ROMP), on the other hand, is employed to generate polymers from cyclic, unsaturated monomers.

Ruthenium-based Grubbs-type complexes represent one of the two main classes of catalysts that have come to dominate the field of OM. There have been numerous modifications made to these systems over the years in order to make them more stable, as well as more selective. Additionally, significant attention has been paid to generating more active Grubbs-type OM catalysts, generally through the incorporation of one or two labile ligands. These fast-initiating species include Grubbs 3^{rd} generation catalysts, trifluoromethanesulfonamide complexes from the Hong group, N-Grubbs-Hoveyda-type systems from Plenio and co-workers, four coordinate compounds from the Piers group, as well as many other examples. One commonality that all of these fast-initiating species share, and nearly all ruthenium-based OM catalysts in general, is a similar canonical structure when it comes to the active catalytic species. It is commonly accepted that the ligand trans to the N-heterocyclic carbene (NHC) or phosphine donor undergoes a ligand substitution reaction with the alkene substrate generating an intermediate with two anionic ligands, a phosphine or NHC ligand, a carbene donor, and a coordinated olefin (Scheme). Although there are a few ruthenium-based OM pre-catalysts that diverge significantly from the classic Grubbs-type structure, these complexes are still thought to form the same general active species shown in Scheme, in situ. As such, there is still a large amount of room for innovation within the field OM, and the opportunity to unlock new reaction manifolds through the development of ruthenium-based systems that access alternative catalytic pathways.

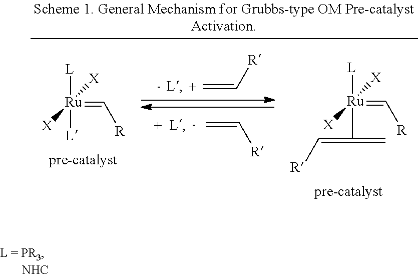

Phosphinimines are an underutilized class of ligands for late-transition metals that are beginning to receive more and more attention, especially as chelating donors for catalytic applications. Monodentate phosphinimines, however, are quite rare on ruthenium, whereas monodentate phosphinimide complexes are a bit more common. Moreover, while phosphinimides have been utilized in early transition metal-based alkyne metathesis, phosphinimines and phosphinimides have not been used in ruthenium-based OM catalysts.

### SUMMARY OF THE INVENTION

The present invention is broadly concerned with catalyst materials for use in olefin metathesis reactions, as well as methods of synthesizing and using the same.

In one embodiment, there is provided a method for catalyzing a metathesis reaction comprising contacting an olefin reactant with a catalyst material comprising a ruthenium phosphinimine complex.

In another embodiment, there is provided a material for catalyzing an olefin metathesis reaction comprising a ruthenium phosphinimide complex.

In another embodiment, there is provided a method for synthesizing a ruthenium phosphinimine complex. The method comprises reacting a source of phosphinimine or phosphinimide ligands with a Grubbs catalyst to produce the ruthenium phosphinimine complex.

### BRIEF DESCRIPTION OF THE DRAWINGS

Figure (FIG. 1A and FIG. 1B are graphs showing kinetic studies investigating the homocoupling of 1-hexene using Ru1, with FIG. 1A showing different concentrations of 1-hexene at 25° C. (0.0021 M Ru1), with FIG. 1B showing the rate dependence on 1-hexene concentration at 25° C.

FIG. 2A and FIG. 2B are graphs showing kinetic studies investigating the homocoupling of 1-hexene using Ru1, with FIG. 2A showing different concentrations of Ru1 at 25° C. (5 M 1-hexene), with FIG. 2B showing the rate dependence on Ru1 concentration at 25° C.

FIG. 3A and FIG. 3B are graphs showing kinetic studies investigating the homocoupling of 1-hexene using Ru1, with FIG. 3A showing at different temperatures (0.0021 M Ru1 and 5 M 1-hexene), with FIG. 3B showing activation parameters using an Eyring plot.

FIG. 4 is a diagram showing the free energies of tricyclohexylphosphine and triphenylphosphinimine (left) versus triphenylphosphine and tricyclohexylphosphinimine (right).

FIG. 5 is a ORTEP3 representation (thermal ellipsoids at 50% probability) and atom numbering for Ru1, where most of the hydrogens and two other complexes that were part of the asymmetric unit were removed for clarity.

FIG. 6 is a graph showing the reaction profile for the homocoupling of 1-hexene using Ru1 (1:2400 Ru1: 1-hexene at 25° C.).

FIG. 7A, FIG. 7B, and FIG. 7C are graphs showing kinetic studies investigating the homocoupling of 1-hexene using Ru1, with FIG. 7A showing the rate dependence on 1-hexene concentration at 25° C., with FIG. 7B showing the rate dependence on Ru1 concentration at 25° C., with FIG. 7C showing an Eyring plot used to determine activation parameters.

FIG. 8 is a diagram showing the free energy profiles of the two possible OM pathways where the phosphinimine ligand de-coordinated (black) or the phosphine ligand de-coordinated (grey).

FIG. 9 is a graph showing the reaction profiles for the homocoupling of 1-hexene using Ru1 (1:2400 Ru1: 1-hexene at 25° C.) under various conditions.

FIG. 10A and FIG. 10B are graphs showing reaction profiles for the homocoupling of 1-hexene using Ru1 (1:2400 Ru1: 1-hexene at 25° C.), where after 30 minutes, an additional 2400 equiv. of 1-hexene were added (FIG. 10A) and 1 equiv. of Ru1 was added (FIG. 10B).

FIG. 11 is a ORTEP3 representation (thermal ellipsoids at 50% probability) and atom numbering for Ru2, where most of the hydrogens were removed for clarity.

FIG. 12 is a ORTEP3 representations (thermal ellipsoids at 50% probability) of 1a(PCy₃) (left) and 1a(SIMes) (right) where most of the hydrogens were removed for clarity.

### DETAILED DESCRIPTION

The present invention is concerned with improved catalyst materials for olefin metathesis reactions. As used herein, the term “catalyst” refers to a substance that increases the rate of a chemical reaction without itself undergoing any permanent chemical change. The term “catalyst” as used herein may include materials that are considered “pre-catalyst” materials or “catalyst precursors,” which include substances that are converted to a catalyst (i.e., activated) during the course of the catalyzed reaction. In certain embodiments, the catalyst materials are particularly suitable for catalyzing olefin metathesis reactions, as described in greater detail herein.

The catalyst materials generally comprise a ruthenium phosphinimine complex. As used herein, the term “ruthenium phosphinimine complex” refers to a coordinated compound comprising at least one phosphinimine ligand bonded to a central ruthenium metal atom. Phosphinimine ligands have a general chemical formula of NHPR₃, where the three R groups can be a variety of substituent groups, which are typically organic substituent groups. With respect to catalyst initiation, it has been discovered that phosphinimines are particularly advantageous ligands because of their umbrella-like structure, which is notably distinct from phosphines or NHCs. Without being bound by any theory, it is believed that phosphinimines therefore create more space directly at the metal, potentially facilitating alkene binding and promoting associative or interchange mechanisms of pre-catalyst activation, while still providing significant steric protection to reactive intermediates. The metal-ligand bond strengths of the targeted complexes can be tuned through variations in the donating ability of the phosphinimine moiety.

In certain embodiments, the ruthenium phosphinimine complex has the structure of formula (I) or (II), below:

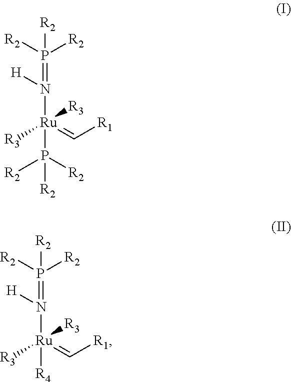

where each R₁, R₂, R₃, and R₄ is independently selected from the group consisting of the possible options listed in the table below

| Group | Possible substitutions |
|---|---|
| R₁ | alkyls (e.g., C1-C15), aromatic rings |
| | (e.g., with size from 4-10 carbons), |
| | heterocyclic rings (e.g., with size 4-10 atoms), |
| | aromatic or heterocycle rings with substitutions. |
| R₂ | alkyls (e.g., C1-C15), aromatic rings |
| | (e.g., with size from 4-10 carbons), |
| | heterocyclic rings (e.g., with size 4-10 atoms), |
| | aromatic or heterocycle rings with substitutions. |
| R₃ | halides, cyanide, aryloxides, alkoxides, |
| | fluoroalkoxides, thiolates, pyrrolides, |
| | carboxylates, sulfates, phosphates, nitrates. |
| R₄ | heterocyclic rings (e.g., with size 4-10 atoms), |
| | heterocycle rings with substitutions. |

In certain embodiments, R₁ is a phenyl group.

In certain embodiments, each R₂ is independently selected from the group consisting of phenyls, ethyls, cyclohexyls, and tert-butyls. For example, the phopshinimine ligand include one of the following structures:

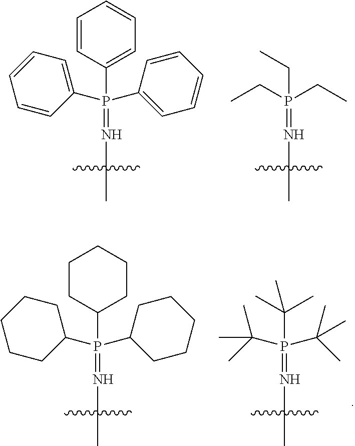

In certain embodiments, each R₃ is independently selected from the group consisting of triflate, chloride, alkoxides, fluoroalkoxides, thiolates, and pyrrolides. For example, R₃ may include one of the following structures:

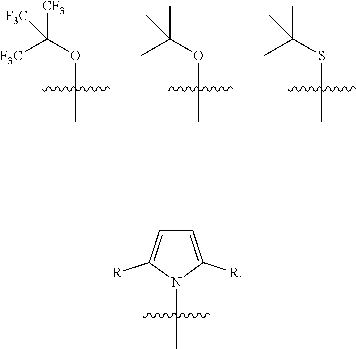

In certain embodiments, R₄ is an N-heterocyclic carbene (SIMes). For example, R₄ may include the following structure:

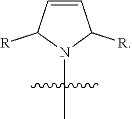

Referring again to formulas (I) and (II) above, in certain embodiments, the ruthenium phosphinimine complex may have a Ru—N bond length (i.e., the distance between the atoms) of less than about 2.30 Å. In certain embodiments, the ruthenium phosphinimine complex may have a Ru—N bond length of about 2.00 Å to about 2.25 Å, or about 2.05 Å to about 2.15 Å.

In certain embodiments, the ruthenium phosphinimine complex may have a Ru—P bond length of not more than about 2.35 Å. In certain embodiments, the ruthenium phosphinimine complex may have a Ru—P bond length of about 2.20 Å to about 2.35 Å.

In certain embodiments, the ruthenium phosphinimine complex may have a Ru—C bond length of at least about 1.80 Å. In certain embodiments, the ruthenium phosphinimine complex may have a Ru—C bond length of about 1.80 Å to about 2.10 Å, or about 2.02 Å to about 2.08 Å.

In certain embodiments, the ruthenium phosphinimine complex may have a P—N bond length of about 1.25 Å to about 1.75 Å, or about 1.50 Å to about 1.65 Å.

In certain embodiments, the ruthenium phosphinimine complex does not include an N-Heterocyclic Carbene (NHC) or phosphine ligand.

In certain embodiments, the ruthenium phosphinimine complex can be synthesized from a Grubbs catalyst starting material, such as the Grubbs Generation I catalyst. The Grubbs catalyst can be dissolved in an organic solvent (e.g., benzene), while a source of phosphinimine or phosphinimide ligands (e.g., Ph₃PNLi) is separately dissolved in another solvent (e.g., THF). These separate solutions can then be mixed to initiate the ligand substitution reactions. In certain embodiments, silver nitrate can be added to the mixture, which can improve the synthesis reaction. After mixing for at least about an hour, ruthenium complexes comprising one or more phosphinimine ligands is formed, and the resulting solution can then be filtered and/or dried or otherwise recovered. The resulting catalyst material (i.e., the ruthenium phosphinimine complex) generally be in the form of a powder, which may be used as recovered in powder form or further processed before use. For example, the ruthenium phosphinimine complex powder may be deposited or otherwise applied onto a solid substrate or formed into particles that can be used in a fixed or fluidized bed reactor.

Methods in accordance with embodiments of the present invention are directed to catalyzing metathesis reactions, and particularly, olefin metathesis reactions. In certain embodiments, the methods generally comprise contacting an olefin reactant with a catalyst material comprising a ruthenium phosphinimine complex, as described herein. As used herein, the term “olefin reactant” refers to one or more allkene molecules that undergoes a metathesis reaction when exposed to a catalyst material described herein. In certain embodiments, the olefin reactant may comprise one or more terminal alkenes. Exemplary terminal alkenes include, but at not limited to, 1-hexene, 5-decene, 4-methyl-1-pentene, 3-methylpent-1-ene, and 3,3-dimethyl-1-butene. However, in certain embodiments, sterically encumbered (e.g., 3,3-dimethyl-1-butene) and/or electron-deficient olefins are avoided. Exemplary olefin reactants, particularly for ROMP type reactions include, but are not limited to, norbornene, dicyclopentadiene, cyclooctene, cycloheptene, 5,6-di-(carbomethoxy)-norbornene. Exemplary olefin reactants, particularly for CM type reactions include, but are not limited to, hex-5-en-2-one, allyl acetate, allyl amine, allyl alcohol, 3-butenyl acetate, styrene, para-methylstyrene, and para-methoxystyrene, para-fluorostyrene and para-nitrostyrene, 5-decene. A variety of other alkenes may also be used as an olefin reactant in accordance with embodiments of the present invention.

In certain embodiments, the olefin reactant is contacted with the catalyst material by introducing the olefin reactant into a reactor in which the catalyst materials reside, thereby converting at least a portion of the olefin reactant into an olefin product that is different from the olefin reactant. The olefin reactant may be introduced to the reactor as a substantially pure feedstock or as a mixture with other components (e.g., other hydrocarbons, water, etc.). The olefin reactant or feedstock may be introduced or reacted as a liquid, a gas, or two-phase stream.

The reactor may be a batch or continuous reactor, and the particular type and size may be selected depending on the particular application (e.g., starting materials and desired products). In certain embodiments, the reactor is a fixed bed or fluidized bed reactor. In certain embodiments, the catalyst materials may be dissolved or suspended in a solvent prior to being contacted with the olefin reactant. In certain embodiments, the olefin reactant is introduced into the reactor at a weight ratio of about 1:10 to 1:250,000, about 1:20 to about 1:75,000, about 1:100 to about 1:48,000, or about 1:1,000 to 1:25,000 catalyst-to-reactant. However, in certain embodiments, the catalyst materials described herein are capable of catalyzing the metathesis reactions of greater amounts of olefins as compared to prior catalyst materials. Additionally, in certain embodiments, metathesis reaction is catalyzed without an induction (or activation) period of the catalyst (i.e., no measurable delay in activity). The catalyst materials are suitable for use at a wide range of reaction temperatures. For example, in certain embodiments, the olefin reactant is contacted with the catalyst materials and reacted at a temperature of about −100° C. to about 100° C., about −75° C. to about 60° C., about −25° C. to about 50° C., or about 0° C. to about 40° C.

Upon contacting the catalyst materials, at least a portion of the olefin reactant undergoes a metathesis reaction. For example, at least a portion of the olefin reactant may undergo cross metathesis (CM), which may include homocoupling and/or heterocoupling reactions, ring opening metathesis polymerization (ROMP), ring closing metathesis (RCM), and/or acyclic diene metathesis polymerization (ADMET). Regardless the particular metathesis reaction, at least a portion of the olefin reactant is converted into an olefin product that is different than the olefin reactant. The particular product(s) formed will depend on a variety of factors, such as the particular olefin reactants, reaction conditions, and catalyst variations.

The overall conversion will also depend on a variety of factors, such as those listed above, as well as residence time. However, in certain embodiments, after contacting the olefin reactant with the catalyst materials for at least about 10 seconds, at least about 15 seconds, at least about 30 seconds, at least about 1 minute, at least about 10 minutes, at least about 30 minutes, at least about 1 hour, at least 2 hours, or at least about 24 hours (e.g., residence time of the reaction), at least about 30%, at least about 40%, at least about 50%, at least about 60%, at least about 70%, at least about 80%, at least about 90%, at least about 95%, or at least about 99% conversion can be achieved. In certain embodiments, after contacting the olefin reactant with the catalyst materials for about 10 seconds to 1 minute (e.g., residence time of the reaction), at least about 30%, at least about 40%, at least about 50%, at least about 60%, at least about 70%, at least about 80%, at least about 90%, at least about 95%, or at least about 99% conversion can be achieved particularly for linear polymers and not cross-linked polymers. In certain embodiments, the olefin product produced by the reaction can have alternating groups (i.e., linear alkane and cyclic alkane moieties) of at least about 50%, at least about 60%, at least about 70%, at least about 80%, or at least about 85% alternating linkages. In certain embodiments, the olefin product produced by the reaction can have a trans or E selectivity (i.e., a trans alkene reaction product) of at least about 50%, at least about 60%, at least about 70%, at least about 80%, at least about 90%, at least about 95%, or at least about 99%.

Advantageously, the mechanism of the catalyst materials described herein is different than known catalysts because the phosphinimine stays bound to the metal center, and the trans ligand decoordinates. This achieves improved reaction conversion and in less time that prior catalysts. This mechanism also achieves preferred selectivity. For example, complete reactions to 100% conversion can be achieved, in certain embodiments, within as little as 10 or 15 seconds. Additionally, the catalyst materials can be used to produce alternating ROMP copolymers in 2 to 48 hours with 30-60% conversion including 75-85% alternating diads. When desired, the catalyst materials and reaction conditions may also be tuned to achieve up to about 50% or up to about 60% cis or Z selectivity.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods and compositions in accordance with the invention. It is to be understood, however, that these examples are provided by way of illustration and nothing therein should be taken as a limitation upon the overall scope of the invention.

### Example I

### INTRODUCTION

Phosphinimines are an underutilized class of ligands for late-transition metals that are beginning to receive more and more attention, especially as chelating donors for catalytic applications. While phosphinimides have been utilized in early transition metal-based alkyne metathesis, to the best of our knowledge, phosphinimines and phosphinimides have not been used in ruthenium-based OM catalysts. These ligands are intriguing due to their unique electronic structure; they can act as both strong σ- and π-donors due to their ylide-like structure. In this example, the synthesis of a ruthenium phosphinimine complex and its characterization are presented. Furthermore, the catalytic activity of the phosphinimine pre-catalyst for the homocoupling of terminal olefins is explored, including kinetic studies, mechanistic investigations, decomposition studies, and a brief substrate scope.

### Experimental

All procedures and manipulations were performed under a nitrogen atmosphere using standard Schlenk-line and glovebox techniques unless stated otherwise. Solvents were dried and deoxygenated under argon using a LC Technology Solutions Inc. SP-1 stand-alone solvent purification system. All alkenes were dried and distilled over CaH₂, then stored over activated molecular sieves under a nitrogen atmosphere. Deuterated solvents were purchased from Cambridge Isotope Laboratories, Sigma-Aldrich, Acros Organics, or Alfa Aesar degassed, dried over CaH₂, and stored over activated molecular sieves prior to use unless stated otherwise. Triphenylphosphinimine (Ph₃PNH) was synthesized according to a literature procedure. All other reagents were purchased from commercial sources and utilized without further purification unless stated otherwise. NMR spectra were recorded at ambient temperature and pressure using a Bruker 400 MHz spectrometer (400 MHz for 1H, 162 MHz for 31P, and 100 MHz for 13C). The 1H and 13C NMR spectra were measured relative to partially deuterated solvent peaks but are reported relative to tetramethylsilane (TMS). All 31P chemical shifts were measured relative to 85% phosphoric acid as an external reference. Spectra were processed and visualized with MestReNova v12.0.0-20080. The elemental analyses were performed at the University of Rochester, Department of Chemistry, on a PerkinElmer 2400 Series II Analyzer. Single crystal X-ray data were collected using a Rigaku XtaLAB Synergy-S diffractometer equipped with a HyPix-6000HE Hybrid Photon Counting (HPC) detector and dual Mo and Cu microfocus sealed X-ray source (Cu Ka λ=1.54184 Å). The data collection strategy was calculated using CrysAlisPro to ensure desired data redundancy and percent completeness. Unit cell determination, initial indexing, data collection, frame integration, Lorentz-polarization corrections and final cell parameter calculations were carried out using CrysAlisPro. An absorption correction was performed using the SCALE3 ABSPACK scaling algorithm embedded within CrysAlisPro. The structures were solved using the ShelXT structure solution program using Intrinsic Phasing and refined by Least Squares using the ShelXL program. All non-hydrogen atoms were refined anisotropically. Most hydrogen atom positions were calculated geometrically and refined using the riding model. Olex2 was used for the preparation of the publication materials. All crystals were mounted in inert oil and transferred to the cold gas stream of the diffractometer.

Synthesis of Lithium Triphenylphosphinimide (Ph₃PNLi). Inside of a glovebox, a suspension of 0.500 g Ph₃PNH (1.80 mmol, 1 equiv.) in approximately 10 mL diethyl ether was cooled to −30° C. The suspension was then treated with 1.24 mL 1.6 M ⁿBuLi/hexanes (1.98 mmol, 1.1 equiv.), which was added dropwise with stirring. The reaction mixture was left stirring for 48 h, and the white precipitate was isolated by filtration, washed with ether (4×5 mL), as well as benzene (1×2 mL). The precipitate was then dried under reduced pressure. Yield: 0.460 g (90.1%). ¹H NMR (400 MHZ, CD₂Cl₂) δ: 7.80 (dd, 6H, Aromatic-CH, J=12.0, 7.6 Hz) 7.59-7.52 (m, 3H, Aromatic-CH), and 7.51-7.42 (m, 6H, Aromatic-CH) ppm. ³¹P {¹H} NMR (162 Hz, CD₂Cl₂) δ: 21.65 (s) ppm. ¹³C NMR (100 MHZ, CD₂Cl₂) δ: 134.34 (d, Aromatic-C, J=94.8 Hz), 131.92 (d, Aromatic-CH, J=9.5 Hz), 131.23 (d, Aromatic-CH, J=2.8 Hz), and 128.30 (d, Aromatic-CH, J=11.6 Hz) ppm. Anal. Calcd for C₁₈H₁₅PNLi: C, 78.23; H, 5.48; N, 5.07; Found: C, 64.38; H, 4.92; N, 4.05. CHN analytical experiments were performed multiple times across several different samples and similar data were obtained each time. The discrepancy between theoretical and experimental CHN analytical data is hypothesized to be due to the air sensitive nature of the compound.

Synthesis of Complex Ru1 [Ru(CHPh)(Ph₃PNH)(PCy₃)Cl₂]. Inside of a glovebox, a solution of 0.200 g Grubbs Generation I catalyst (0.243 mmol, 1 equiv.) in approximately 10 mL benzene was prepared. To this was added a solution of 0.069 g Ph₃PNLi (0.243 mmol, 1 equiv.) in approximately 4 mL of THF with stirring. The solution turned red/orange in color. The reaction mixture was stirred for 5 min. and then added to a suspension of 0.041 g AgNO₃ (0.243 mmol, 1 equiv.) in approximately 4 mL THF in the dark (the vial was wrapped in aluminum foil). The color of the reaction mixture turned dark yellow gradually. The solution was stirred for 1 h and then filtered through Celite. The solvent was removed under reduced pressure, and the resulting residue was stirred in approximately 6 mL pentane for 30 min. The precipitate was isolated by filtration and washed with pentane until the filtrate was colorless. After drying under reduced pressure, the precipitate was redissolved in approximately 6 mL of benzene and filtered through Celite. The solvent was evaporated under reduced pressure to give a dark residue, which was stirred in approximately 5 mL pentane for 1 h, and then the solid product was isolated by filtration. The yellowish green powder was washed with pentane until the filtrate was colorless and then dried under reduced pressure. Yield: 0.097 g (48.7%). Crystals suitable for single crystal X-ray diffraction studies were grown from a concentrated toluene solution of Ru1 cooled to −30° C. ¹H NMR (400 MHZ, C₆D₆) δ: 19.71 (d, 1H, Ru=CHPh, J=8.6 Hz), 8.81 (d, 2H, Aromatic-CH, J=7.7 Hz), 7.59-7.49 (m, 6H, Aromatic-CH), 7.39 (t, 1H, Aromatic-CH, J=7.3 Hz), 7.24 (t, 2H, Aromatic-CH, J=7.6 Hz), 7.01-6.93 (m, 3H, Aromatic-CH), 6.93-6.86 (m, 6H, Aromatic-CH), 4.84 (dd, 1H, P=NH, J=6.7, 2.5 Hz), 2.65 (q, 3H, Aliphatic-CH, J=12.1 Hz), 2.14-2.01 (m, 6H, Aliphatic-CH), 1.86-1.67 (m, 12H, Aliphatic-CH), 1.65-1.55 (m, 3H, Aliphatic-CH), and 1.29-1.19 (m, 9H, Aliphatic-CH) ppm. ³¹P {¹H} NMR (162 Hz, C₆D₆) δ: 53.18 (s), and 35.49 (s) ppm. ¹³C NMR (100 MHZ, C₆D₆) δ: 153.73 (s, Aromatic-C), 132.88 (d, Aromatic-CH, J=10.2 Hz), 131.46 (d, Aromatic-CH, J=3.0 Hz), 130.32 (d, Aromatic-C, J=100.6 Hz), 129.49 (s, Aromatic-CH), 128.94 (s, Aromatic-CH), 33.85 (d, Aliphatic-CH, J=21.6 Hz), 29.91 (s, Aliphatic-CH), 27.81 (d, Aliphatic-CH, J=10.4 Hz), and 26.66 (s, Aliphatic-CH) ppm. Anal. Calcd for C₄₃H₅₅Cl₂NP₂Ru: C, 63.00; H, 6.76; N, 1.71; Found: C, 62.74; H, 6.64; N, 2.04.

Computational details. Density functional theory (DFT) calculations were performed using the GAMESS (US) package (2016 R1) and the M11-L (catalytic cycle) or B3LYP (Ph₃PNH versus Cy₃PNH) hybrid functionals. Ruthenium was treated with the Def2-TZVP relativistic effective core potential and associated basis set, while all other atoms were treated with the 6-311++G (d,p) basis set. A tight integration grid where NRAD=99 and NLEB=1202 was used throughout for increased accuracy. Ground states were connected to their transition states by performing intrinsic reaction coordinate (IRC) calculations. Odd-electron species were not considered, and stationary points were characterized by normal-mode analysis. Full vibrational and thermochemical analyses (1 atm, 298 K) were performed on optimized structures to obtain free energies (G°) and enthalpies (H°). Optimized ground states were found to have zero imaginary frequencies, while transition states were found to have one imaginary frequency. Three dimensional visualizations of calculated structures were generated using MacMolPlt.

Kinetic Studies with Ru1 and I-hexene. Inside of a glovebox, a solution of CDCl₃ (between 1.59 mL and 0.40 mL) and 1-hexene (between 0.0421 g and 0.842 g) was prepared and capped. Separately, a stock solution of Ru1 was prepared by dissolving 31.4 mg of the pre-catalyst in 3.2 mL (4.768 g) CDCl₃. Outside of a glovebox, the Ru1 stock solution (between 0.088 and 0.35 mL) was added to the 1-hexene solution (in air) at 25° C. All catalytic reactions had a final total volume of 2 mL, with 1-hexene concentrations of 0.25, 1.25, 2.5, or 5 M, and Ru1 concentrations of 2.09, 1.57, 1.05, or 0.52 mM. The progress of each reaction was monitored over two hours, with 0.05 mL samples taken at 1, 2, 3, 5, 10, 15, 30, 45, 60, and 120 min., quenching in 0.5 mL of 12 mol % (with respect to catalyst) acetic acid in CDCl₃. Conversions were determined using ¹H NMR spectroscopy according to literature methods. To determine activation parameters, the solutions of 1-hexene and CDCl₃ were first left stirring at a specific temperature (40° C., 25° C., 0° C., or −18° C.) outside of a glovebox in order to equilibrate before adding the Ru1 stock solution.

Stability Studies with Ru1 and I-hexene. For Case I, the catalytic reactions were carried out the exact same way as the kinetic studies outlined above, with a 5 M 1-hexene concentration and a 2.1 mM Ru1 concentration (all solvents were dried and degassed). For Case II, a very similar procedure to Case I was utilized, except that 1-hexene and CDCl₃ (used as received from the chemical supplier) were utilized to generate the initial I-hexene solution outside of a glovebox. Then, the Ru1 stock solution was added (also outside of a glovebox) to initiate catalysis. For Case III, the catalytic reactions were carried out the exact same way as Case I, except that the reaction was never taken out of a glovebox. Lastly, for Case IV, 1-hexene and CDCl₃ were prepared using several freeze-pump-thaw cycles, but were not dried over chemical reagents or molecular sieves. These were then used to generate the initial 1-hexene solution inside of a glovebox, following the same procedure as Case I, and then the Ru1 stock solution was added (also inside of a glovebox) to initiate catalysis. It should be noted that degassed acetic acid was used to generate the quenching solutions for Cases III and IV.

Catalytic Reactions with Two Additions of 1-hexene. Following a procedure very similar to Case I, as well as the kinetic studies outlined above, reactions with a 5 M 1-hexene concentration and a 2.1 mM Ru1 concentration were performed outside of a glovebox with dried and degassed solvents/reagents. After 30 min., an additional 0.842 g of 1-hexene was added to the catalytic mixture and the reaction was monitored for another 120 min. Separately, following a procedure very similar to Case III, reactions with a 5 M 1-hexene concentration and a 2.1 mM Ru1 concentration were performed inside of a glovebox with dried and degassed solvents/reagents. After 30 min., an additional 0.842 g of 1-hexene was added to the catalytic mixture and the reaction was monitored for another 120 min.

Catalytic Reactions with Two Additions of Ru1. Following a procedure very similar to Case I, as well as the kinetic studies outlined above, reactions with a 5 M 1-hexene concentration and a 2.1 mM Ru1 concentration were performed outside of a glovebox with dried and degassed solvents/reagents. After 30 min., an additional 0.35 mL of Ru1 stock solution was added to the catalytic mixture and the reaction was monitored for another 120 min. Separately, following a procedure very similar to Case III, reactions with a 5 M 1-hexene concentration and a 2.1 mM Ru1 concentration were performed inside of a glovebox with dried and degassed solvents/reagents. After 30 min., an additional 0.35 mL of Ru1 stock solution was added to the catalytic mixture and the reaction was monitored for another 120 min.

General Procedure for Homocoupling of Terminal Olefins using Ru1. A scintillation vial was loaded with substrate and CDCl₃. To this was added 0.51 mL of the Ru1 stock solution outlined above in the kinetic studies (inside of a glovebox), and then sealed. All catalytic reactions had a final total volume of 1 mL, with substrate concentrations of 0.61 M and Ru1 concentrations of 6.1 mM. After stirring 24 h, another equivalent of Ru1 (5 mg) was added to the reaction mixture, and the solution was stirred for an additional 24 h. The progress of each reaction was monitored over 48 h, with 0.05 mL samples taken at 2, 24, and 48 h, quenching in 0.5 mL of 12 mol % (with respect to catalyst) acetic acid in CDCl₃. Conversions were determined using ¹H NMR spectroscopy according to literature methods. For styrene and its para-substituted derivatives, 0.61 M 1,3,5-trimethoxybenzene was used as an internal standard.

Discussion of Ru1 Formation. Without being bound by any theory, it is believed that during the synthesis of Ru1 an equilibrium was formed between G1 and the phosphinimine complex. In support of this hypothesis, when two equivalents of lithium triphenylphosphinimide were used, more Ru1 was formed, but a large amount of unreacted lithium phosphinimide could be detected in solution (as well as some G1). This result also suggested that protonation of the anionic ligand occurred only after it became coordinated to the metal center. Based on literature precedent from the Fogg group, it is likely that the proton source for the phosphinimine ligand is the phenylidene donor. Therefore, during the reaction, a large portion of the G1 starting material was thought to be decomposing (50% for complete conversion to Ru1). Based on these observations, it was anticipated that the addition of one equivalent of silver nitrate would shift the equilibrium towards Ru1 by removing both a chloride and a tricyclohexylphosphine from solution. Interestingly, if G1 was treated with triphenylphosphinimine itself (both in the presence and absence of silver nitrate) no reaction was observed.

Discussion of Ru1 Crystal Structure. The ruthenium center displayed a distorted square pyramidal geometry (τ₅=0.22) with the phenylidene donor in the axial position. The chloride ligands, on the other hand, were trans to one another (C11A-Ru1A-C12A bond angle of) 169.1° in the equatorial plane, as were the tricylcohexylphosphine and triphenylphosphinimine moieties (N1A-Ru1A-P2A bond angle of) 156.0°. Interestingly, one of the chloride ligands as well as the phosphinimine ligand were bent away from the carbene donor, while the other chloride and the bulkier tricyclohexylphosphine had bond angles much closer to the ideal 90°. In addition, all of the equatorial ligand bond angles were relatively close to the ideal bond angle of 90°, but the chlorides were bent slightly closer to the phosphinimine ligand and away from the larger phosphine donor. For notable bond lengths and angles see Table 1.

TABLE 1
Selected Bond Lengths (Å) and Angles (deg) for Ru1 and G1.

| | |
|---|---|
| Ru1 | G1 |

Bond Lengths (Å)

| | | |
|---|---|---|
| Ru(1A)—C(1A) | 1.83(1) | 1.77(2) |
| Ru(1A)—N(1A)^{a} | 2.081(9) | 2.35(1) |
| Ru(1A)—P(2A) | 2.326(2) | 2.38(1) |
| Ru(1A)—C1(1A) | 2.380(2) | 2.34(1) |
| Ru(1A)—Cl(2A) | 2.381(1) | 2.35(1) |
| P(1A)—N(1A) | 1.607(9) | N/A |

Bond Angles (deg)

| | | |
|---|---|---|
| Cl(1A)—Ru(1A)—Cl(2A) | 169.09(9) | 167.2(4) |
| N(1A)—Ru1A—P(2A)^{a} | 156.0(2) | 161.8(4) |
| C(1A)—Ru(1A)—Cl(1A) | 90.1(3) | 105.4(9) |
| C(1A)—Ru(1A)—Cl(2A) | 100.2(3) | 87.3(9) |
| C(1A)—Ru(1A)—N(1A)^{a} | 109.8(4) | 99.1(9) |
| C(1A)—Ru(1A)—P(2A) | 94.2(3) | 98.7(9) |
| N(1A)—Ru(1A)—Cl(1A)^{a} | 88.6(2) | 89.2(4) |
| N(1A)—Ru(1A)—Cl(2A)^{a} | 84.6(2) | 89.8(4) |
| P(2A)—Ru(1A)—Cl(1A) | 93.64(9) | 89.2(4) |
| P(2A)—Ru(1A)—Cl(2A) | 88.23(9) | 87.8(4) |
| Ru(1A)—N(1A)—P(1A) | 140.4(4) | N/A |

^{a}For G1 the listed values corresponded to the bond lengths and angles involving P(1) rather than

N(1A) from Ru1.¹⁹

Discussion of Ru2 Crystal Structure. The ruthenium center displayed a piano stool structure with two chlorides, a triphenylphosphine ligand, and an η⁶-trans-stilbene moiety. The bond distances and angles were all very similar to a related structure, Ru(η⁶-para-cymene)(PPh₃)Cl₂, already reported in the literature. Even the ruthenium-centroid distances matched well, with values of 1.462 and 1.458 Å for Ru(η⁶-para-cymene)(PPh₃) Cl₂ and Ru2, respectively. For notable bond lengths and angles see Table 2.

TABLE 2
Selected Bond Lengths (Å) and Angles (deg) for Ru2.

| | Ru1 |
|---|---|
| | Bond Lengths (Å) |
| Ru(1)—P(1) | 2.344(4) |
| Ru(1)—C1(1) | 2.406(4) |
| Ru(1)—Cl(2) | 2.396(5) |
| C(7)—C(8) | 1.32(3) |
| | Bond Angles (deg) |
| Cl(1)—Ru(1)—Cl(2) | 89.5(2) |
| P(1)—Ru(1)—Cl(1) | 87.8(2) |
| P(2)—Ru(1)—Cl(2) | 87.6(2) |

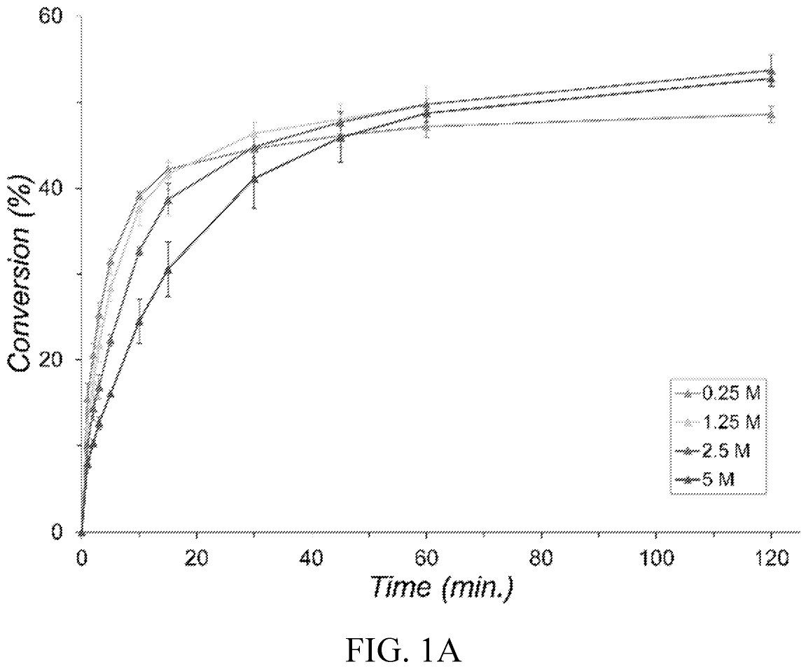

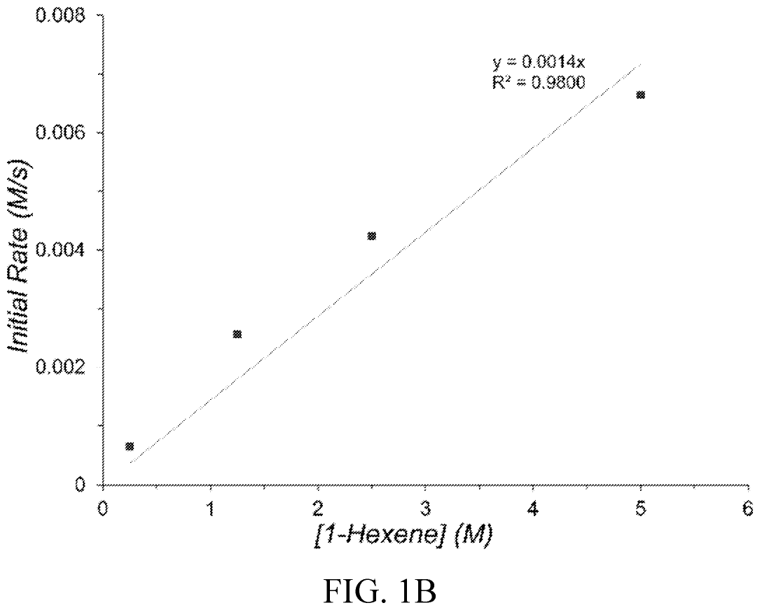

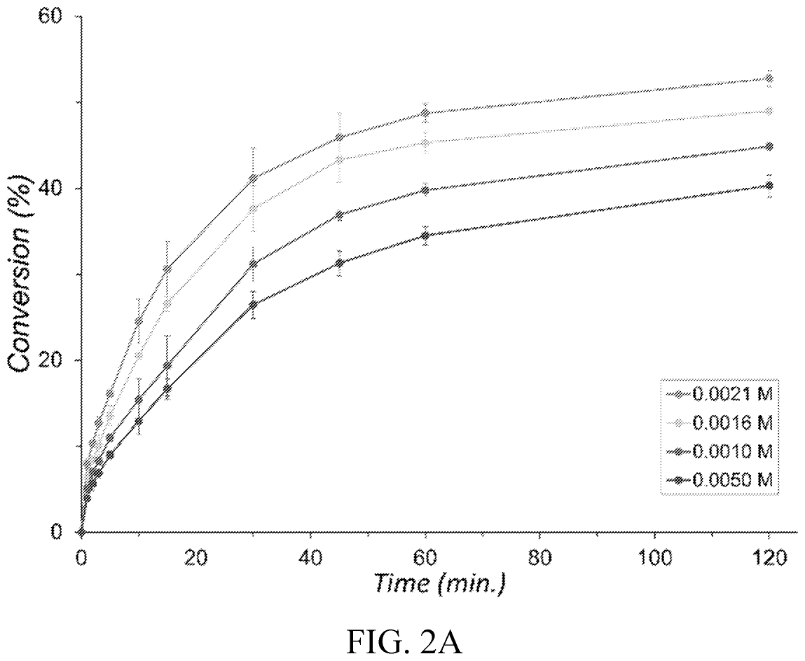

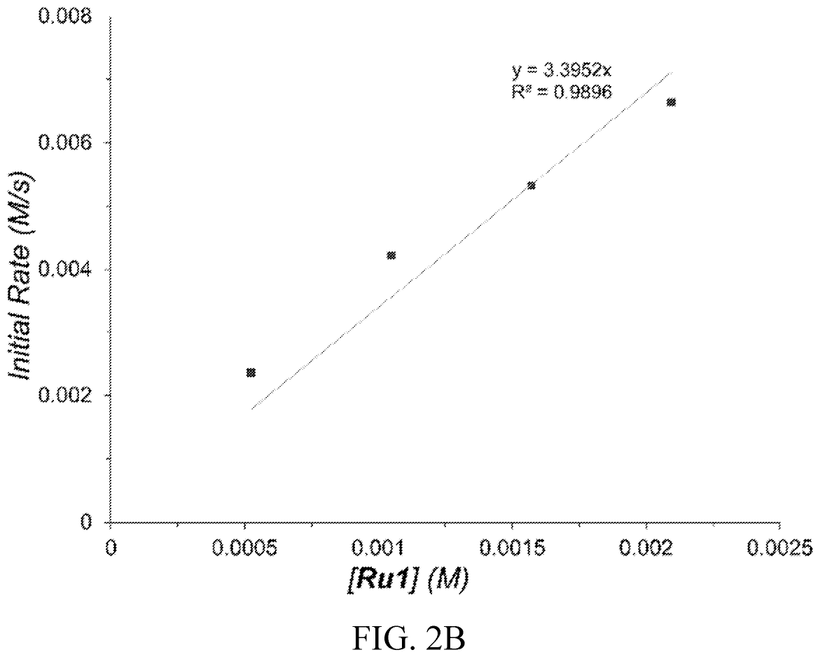

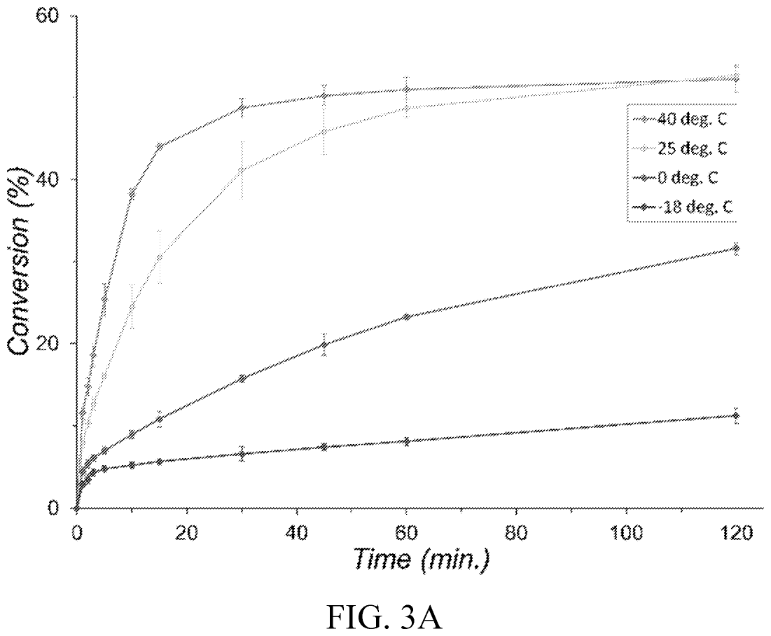

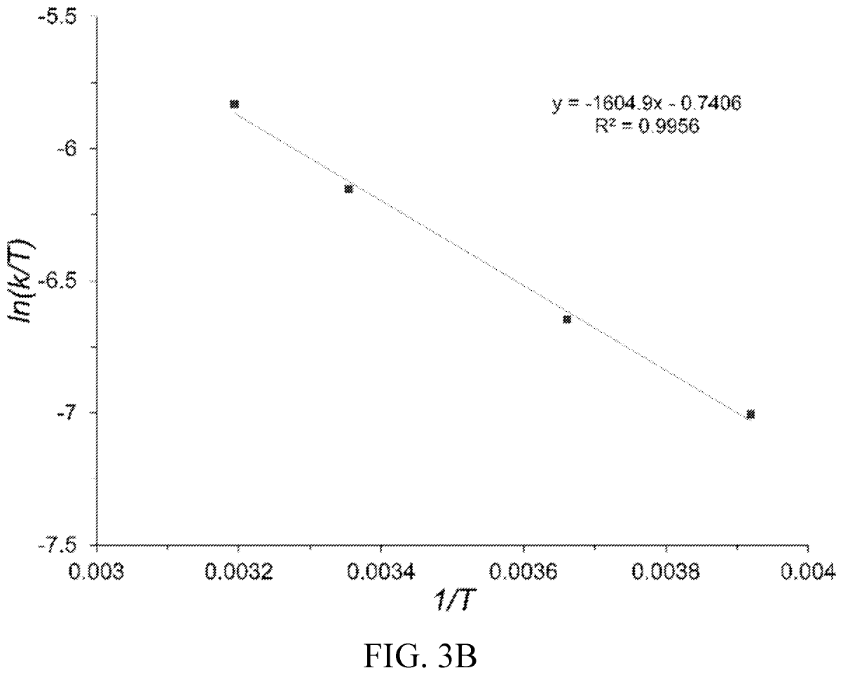

Reaction Profiles and Kinetic Analyses. Reaction rates were determined using the initial rates method (Tables 1-3). Rate constants were calculated from the slope of the least-square fit of plots of reaction rate versus 1-hexene concentration, as well as Ru1 concentration (FIG. 1A, FIG. 1B, FIG. 2A, FIG. 2B). Activation parameters including enthalpy (ΔH^{±}) and entropy (ΔS^{±}) were calculated from the slope and intercept of the least-square fit of an Eyring plot (In k/T vs. 1/T, FIG. 3A and FIG. 3B).

TABLE 3
Initial Rates versus [1-hexene] for
the Homocoupling of 1-hexene using Ru1.

| Initial Rate (M/s) | [1-hexene] (M) |
|---|---|
| 0.006642 | 5 |
| 0.004242 | 2.5 |
| 0.002558 | 1.25 |
| 0.000645 | 0.25 |

TABLE 4
Initial Rates versus [Ru1] for the
Homocoupling of 1-hexene using Ru1.

| Rate (M/s) | [Ru1] (M) |
|---|---|
| 0.006642 | 0.0020945 |
| 0.005325 | 0.0015709 |
| 0.004217 | 0.00104727 |
| 0.002358 | 0.00052364 |

TABLE 5
Initial Rates and Rate Constants versus Temperature
for the Homocoupling of 1-hexene using Ru1.

| T (K) | 1/T | Rate (M/s) | k | In(k/T) |
|---|---|---|---|---|
| 313.15 | 0.0039 | 0.009617 | 0.91831 | −5.8319 |
| 298.15 | 0.0037 | 0.006642 | 0.634233 | −6.15294 |
| 273.15 | 0.0033 | 0.003717 | 0.35493 | −6.64586 |
| 255.15 | 0.0031 | 0.002425 | 0.231559 | −7.00477 |

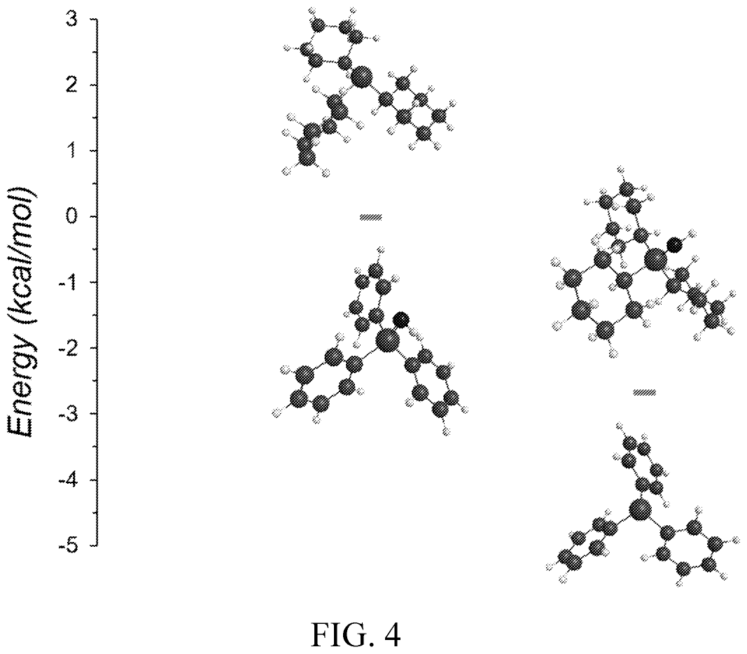

Ground State Energies of Ph₃PNH versus Cy₃PNH. To explore whether or not the exchange of the NH functionality from triphenylphosphinimine to tricyclohexylphosphine was thermodynamically favorable, DFT calculations utilizing the B3LYP functional and 6-311++G** (H, C, P) basis set were performed. The free energies of triphenylphosphinimine and tricyclohexylphosphine were used as the reference point with respect to the free energies of triphenylphosphine and tricyclohexylphosphinimine (FIG. 4). It was found that that the formation of tricyclohexylphosphinimine was thermodynamically favorable with ΔΔG°=−2.7 kcal/mol downhill.

TABLE 6
Selected Crystal Data, Data Collection, and Refinement
Parameters for Compounds Ru1 and Ru2.

| | Ru1•C₇H₈•C₄H₈O | Ru2•0.5(C₅H₁₂) |
|---|---|---|
| empirical formula | C₅₄H₇₁Cl₂NOP₂Ru | C_{34.50}H₃₃ Cl₂PRu |
| FW | 984.02 | 650.55 |
| lattice type | Triclinic | Triclinic |
| space group | P₋₁ | P₋₁ |
| T, K | 140.0(1) | 200.0(1) |
| a, Å | 16.0848(3) | 10.2242(6) |
| b, Å | 16.3004(2) | 11.0571(7) |
| c, Å | 30.2345(4) | 13.8927(9) |
| α, deg | 75.2600(10) | 80.902(5) |
| β, deg | 81.0900(10) | 89.901(5) |
| γ, deg | 84.6530(10) | 76.016(5) |
| V, Å³ | 7561.7(2) | 1503.75(17) |
| Z | 6 | 2 |
| ρ_{calc}/Mg m⁻³ | 1.297 | 1.437 |
| μ(Cu/Mo, Kα) mm⁻¹ | 4.373 (Cu) | 6.515 (Cu) |
| F(000) | 3108 | 666 |
| cryst size, mm³ | 0.184 × 0.038 × 0.035 | 0.1 × 0.1 × 0.1 |
| range θ collected, deg | 2.785 to 72.130 | 3.224 to 77.392 |
| reflns collected/unique | 122597/24931 | 16129/4113 |

| | |
|---|---|
| abs cor | Semi-empirical from equivalents |

| | | |
|---|---|---|
| Max and min transmn coeff. | 1.000 and 0.557 | 1.00000 and 0.88922 |
| goodness of fit | 1.092 | 1.015 |
| R₁(I > 2σ(I))^{a} | 0.0478 | 0.0582 |
| wR₂ (all data)^{a} | 0.1434 | 0.1614 |
| peak and hole, e Å⁻³ | 1.040 and −1.626 | 1.671 and −1.035 |

^{a}Definition of R indices: R₁ = Σ(F_{O} − F_{C})/Σ(F_{O}); wR₂ = [Σ[w(F_{O}² − F_{C}²)²]/[Σ[w(F_{O}²)²]]^{1/2}

### Results and Discussion

Synthesis and Characterization of Ru1. Initial attempts to synthesize a ruthenium-based OM catalyst focused on replacing a chloride ligand in Grubbs first-generation catalyst (G1) with a phosphinimide donor. Lithium triphenylphosphinimide was generated through the deprotonation of triphenylphosphinimine (synthesized according to literature procedures) using one equivalent of nBuLi. G1 was then treated with one equivalent of the anionic ligand, which resulted in a 50:50 mixture of G1 and a new ruthenium phosphinimine complex, according to ¹H NMR spectroscopy (the phosphinimide ligand became protonated). Moreover, free tricyclohexylphosphine was evident in the ³¹P NMR spectrum of the crude reaction mixture. In order to push the reaction to completion, the addition of one equivalent of silver nitrate was used (Scheme 1). A ruthenium phosphinimine complex, Ru1, could then be isolated as a yellow/green powder in good yield (around 49%).

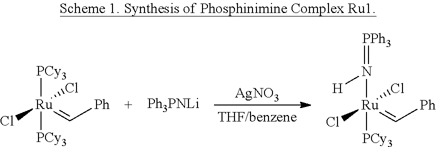

The NMR spectra of Ru1 displayed several characteristic peaks that confirmed the proposed phosphinimine complex had been isolated. The carbene proton was evident as a doublet (coupling to the tricyclohexylphosphine ligand) around 19.7 ppm, slightly upfield shifted from G1. In addition, the aromatic protons for both the phenylidene and triphenylphosphinimine ligands could be detected. Most importantly, however, a broad doublet around 4.8 ppm could be seen, which corresponded to the phosphinimine N—H. With respect to the ³¹P NMR spectrum, the expected signals could be seen: two peaks around 53 and 35 ppm for the two phosphorus-based ligands.

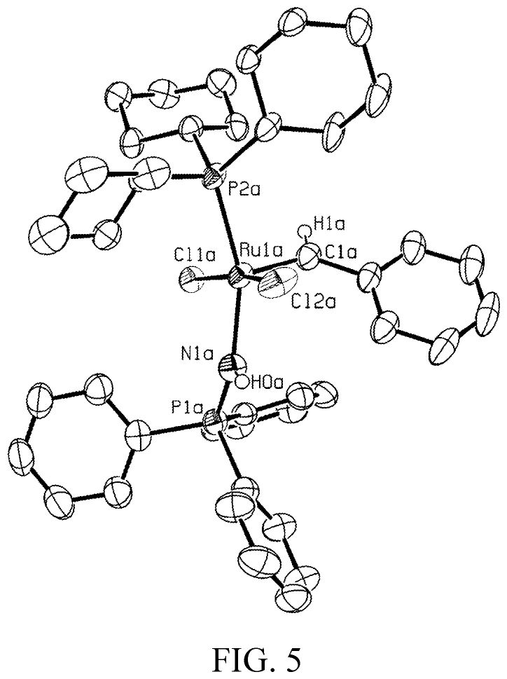

The structure of the pre-catalyst Ru1 was also confirmed by single-crystal X-ray diffraction (FIG. 5). This compound represents the first crystallographically characterized ruthenium carbene species with a monodentate phosphinimine donor. With respect to bond lengths, the Ru1A-P2A distance of 2.326 Å was only slightly shortened in comparison to ruthenium-phosphorus distances reported for G1 (2.38 and 2.35 Å), whereas the Ru1A-Cl1A, Ru1A-Cl2A, and Ru1A-C1A distances were slightly elongated. The ruthenium-phosphinimine (Ru1A-N1A) bond length of 2.08 Å, however, was shorter than the two known ruthenium structures with similar R₃P=N—H donors (2.121 and 2.109 Å). These results were quite surprising, as it was initially believed that the tricyclohexylphosphine would bind much more tightly to the metal center without a trans phosphine donor, and that it would exert a strong trans influence on the triphenylphosphinimine ligand. Based on the values obtained, however, it appears that the opposite occurred: the ruthenium-phosphorus bond was not strengthened considerably, and the phosphinimine donor was tightly bound to the metal. Furthermore, these results suggest that, for these systems at least, the trans influence of triphenylphopshinimine ligand was larger than expected a priori.

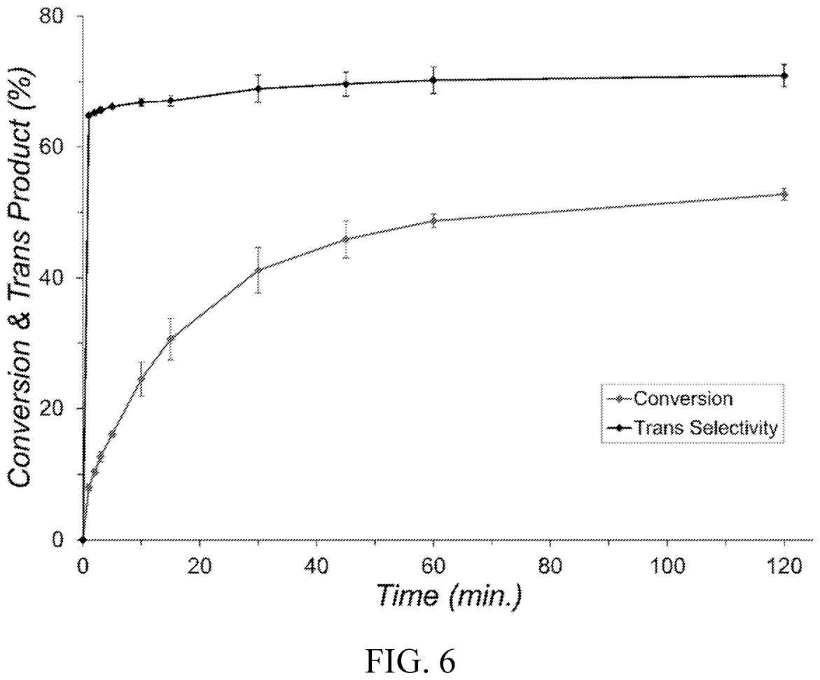

Catalytic Studies Using 1-hexene. Initial experiments evaluating the catalytic efficiency of Ru1 for OM used 1-hexene as a model substrate. Over a 2 h period, 53% 5-decene was obtained as the homocoupled product with 71% trans selectivity (FIG. 6). This is the first report of a ruthenium phosphinimine complex affecting OM. When analyzing the reaction profiles, it immediately became apparent that there was no observable activation period at room temperature, indicating that catalyst initiation was extremely facile. This is particularly notable because many Grubbs-type OM catalysts exhibit a distinct induction period, where ligand de-coordination can be rate-limiting.

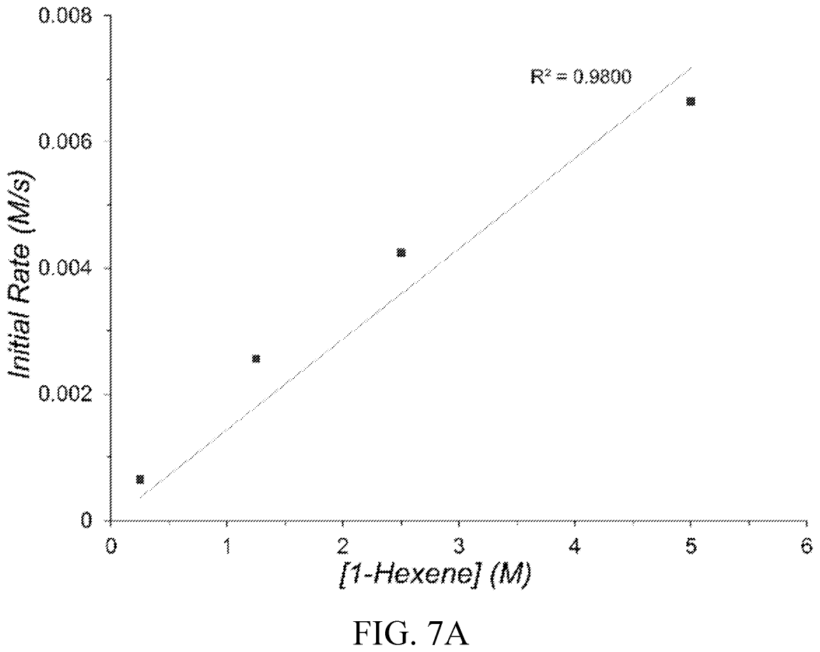

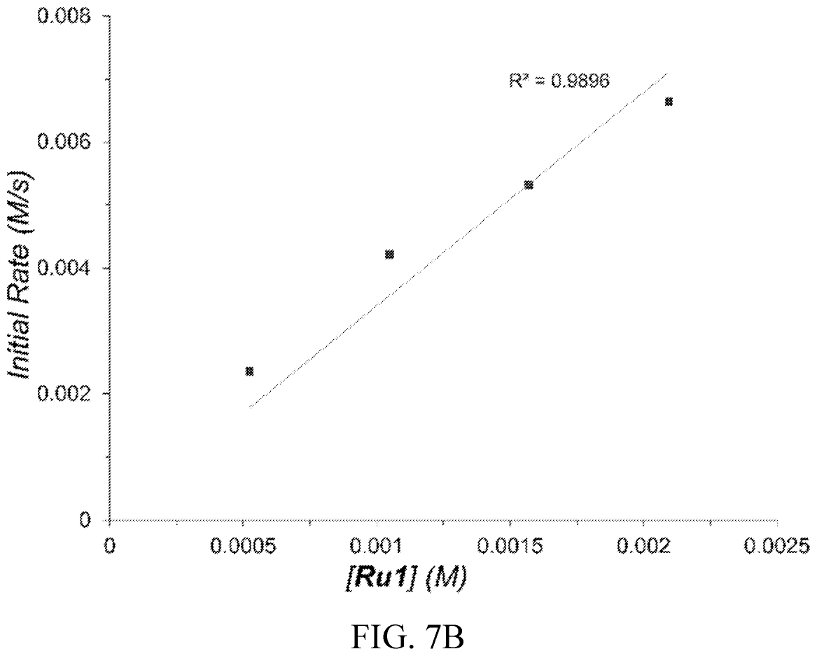

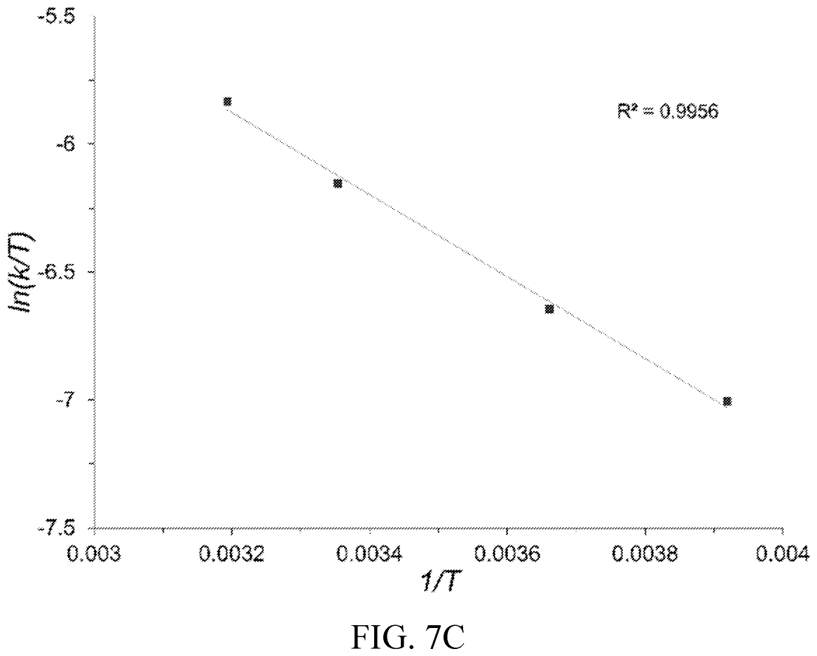

In order to further explore these systems, kinetic investigations were conducted (also utilizing 1-hexene as a model substrate). In particular, the influence of Ru1 concentration and substrate concentration on catalytic activity was assessed using the initial rates method. The empirical rate law was found to have a first-order dependence on both substrate and catalyst, with an experimentally determined rate constant of 0.697±0.050 M⁻¹s⁻¹ (FIG. 7A, FIG. 7B, FIG. 7C). The second order rate law demonstrated that both the substrate and ruthenium complex were involved in the rate-determining step. Moreover, activation parameters for the homocoupling of 1-hexene were obtained from an Eyring plot analysis, with ΔS^{±}, ΔH^{±}, and ΔG^{±}(298 K) values of −48.7±5.1 e.u., 3.19±0.15 kcal/mol, and 17.7 kcal/mol, respectively. In comparison to other fast initiating ruthenium-based OM catalysts, the ΔG^{±}(298 K) for Ru1 is around 0.4 kcal/mol smaller than the ΔG^{±}(298 K) for the Grela nitro-substituted catalyst (18.1 kcal/mol), and around 1 kcal/mol larger than the ΔG^{±}(298 K) for the Blechert-Wakamatsu catalyst (16.7 kcal/mol). Additionally, when comparing the ΔG^{±}(278 K) for Ru1 (17.7 kcal/mol) to the ΔG^{±}(278 K) for Grubbs 3^{rd} generation (15.45 kcal/mol), it is just over 1 kcal/mol larger. Therefore, Ru1 ranks among the fastest initiating ruthenium-based OM catalysts reported in the literature.

When examining the small enthalpy of activation for Ru1 in conjunction with the large negative entropy of activation, this suggested that there was a large decrease in disorder in the rate-limiting step with little to no bond-breaking character. In addition, the values of the experimentally determined activation parameters were consistent with those of a reported system known to undergo an associative ligand substitution reaction. As such, this data, along with the empirical second order rate law, would align with an associative or an associative interchange ligand substitution reaction being the rate-determining step. This result is significant because, as mentioned previously, the rate-limiting step for many Grubbs-type OM catalysts is ligand de-coordination. Moreover, re-coordination of the original trans ligand during catalysis, thus regenerating the initial pre-catalyst, can decrease the concentration of active species in solution. For systems that initiate through a dissociative mechanism, this is particularly difficult to overcome because catalyst activation is independent of olefin concentration, but for Ru1, in contrast, the more substrate that is added, the more efficient the system becomes.

It is possible, however, that there are multiple competing modes of activation. Extensive research on Hoveyda-Grubbs, Grubbs 3^{rd} generation, and a variety of other ruthenium-based OM catalysts has indicated that there may be several competing initiation mechanisms (dissociative, associative, and interchange), depending on a variety of factors, including: the substrate utilized (concentration, steric bulk, electron richness, etc.), the nature of the leaving donor (steric bulk, electron-donating ability, etc.), as well as the overall catalyst structure. Although this may be the case for Ru1, based on the results and activation parameters discussed above (and the lack of saturation kinetics across a large range of substrate concentrations, up to 5 M 1-hexene) an associative or associative interchange ligand substitution is likely the dominant mode of catalyst activation. Without being bound by any theory, it is believed that for Ru1, alkene binding is facilitated by the nitrogen spacer in the triphenylphosphinimine moiety, which places the steric bulk of the ligand further away from the metal center, much like the Stephan group saw for their phosphinimide olefin polymerization catalysts.

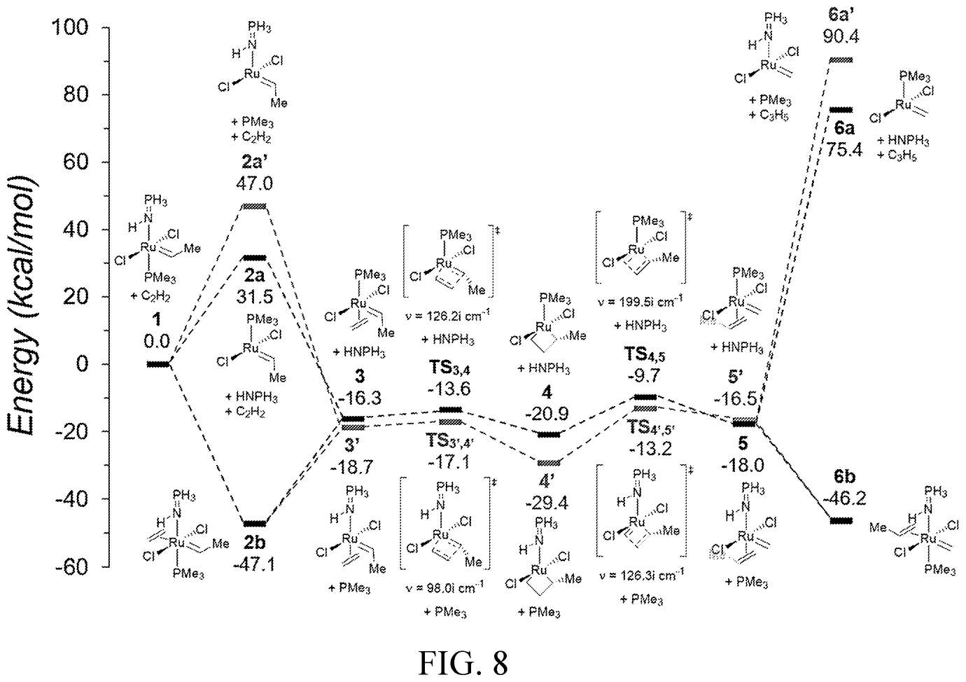

Theoretical and Experimental Mechanistic Studies. To further explore the catalytic mechanism of Ru1, density functional theory (DFT) calculations utilizing the M11-L functional and 6-311++G** (H, C, N, Cl, P)+Def2-TZVP+ECP (Ru) basis sets were performed. A model system with hydrogen atoms in place of the phenyl groups on the phosphinimine donor, and methyl groups in place of the cyclohexyl groups on the phosphine ligand, as well as in place of the substituent on the carbene moiety (to reduce computational cost) was used. Ethylene was utilized as a substrate to model catalyst initiation, metallacyclobutane formation (MCB), and cycloreversion. Two potential catalytic pathways were investigated: 1) the canonical Grubbs-type mechanism where the phosphinimine ligand trans to the phosphine de-coordinated during the catalytic cycle; 2) an alternative mechanism where the phosphine donor trans to the phosphinimine de-coordinated during the catalytic cycle (FIG. 8).

For both the traditional and alternative (labels denoted by ′) pathways, ethylene along with the starting complex 1 (with the phosphine and phosphinimine ligands bound to the metal center) were used as the reference point for the entire catalytic cycle. Then associative and dissociative mechanisms for catalyst initiation were explored. It was found that dissociation of either the phosphine, to give 2a, or phosphinimine, to give 2a′, was associated with a large energetic penalty, with ΔG°₂ₐ=31.5 kcal/mol and ΔG°_{2a′}=47.0 kcal/mol. In contrast, coordination of ethylene to give 2b (same structure for both pathways) was energetically favorable, with ΔG°_{2c}=−47.1 kcal/mol. The greater than 70 kcal/mol or 90 kcal/mol difference between 2c and 2a or 2a′, respectively, along with the kinetic results discussed above give strong experimental and theoretical evidence that catalyst initiation is not proceeding through a dissociative ligand substitution reaction.

In order for catalysis to proceed, de-coordination of one of the phosphorus-containing moieties was needed. The five-coordinate intermediates 3 and 3′ were found to be significantly higher in energy than 2b, however, with ΔG°₃=−16.3 kcal/mol and ΔG°_{3′}=−18.7 kcal/mol. These differences in energy (ΔΔG°=30.8 and 28.4 kcal/mol, respectively) were too large based on the experimentally determined ΔG^{±} for the rate-determining step, and as such, it is believed, without being bound by any theory, that the six-coordinate species 2b is not part of the catalytic cycle. Instead, without being bound by any theory, it is believed that this intermediate is an off-cycle thermodynamic sink. Therefore, the DFT calculations suggest that an associative pathway for catalyst initiation would have too high of a barrier for ligand de-coordination, which points toward an associative-interchange mechanism.

Upon forming 3 and 3′, OM proceeded as expected through the formation of a MCB intermediate, 4 and 4′ (ΔG°₄=−20.9 kcal/mol and ΔG°₅=−29.4 kcal/mol), followed by cycloreversion to generate a new alkene and carbene, 5 and 5′ (ΔG°₅=−18.0 kcal/mol and ΔG°_{5′}, =−16.5 kcal/mol). Transition states for both MCB formation, TS_{3,4} and TS_{3′,4′}, and cycloreversion, TS_{3,4} and TS_{3,4}, were found, all with relatively low energy barriers: ΔG^{±}_{TS3.4}=2.7 kcal/mol, ΔG^{±}=_{TS4.5}=11.2 kcal/mol, ΔG^{±}TS_{3′,4′}=1.6 kcal/mol, and ΔG^{±}TS_{4′,5′}=16.2 kcal/mol. Lastly, the mechanism of alkene substitution was examined. As seen previously during catalyst initiation, olefin de-coordination to form a four-coordinate intermediate, 6a and 6a′, was highly disfavored with an extremely large energetic penalty; ΔG°₆ₐ=75.4 kcal/mol and ΔG_{6a′}=90.4 kcal/mol. Re-coordination of the phoshpinimine or the phosphine ligand, 6b (same structure for both pathways), on the other hand, was energetically downhill (ΔG°_{6b}=−46.2 kcal/mol) and much like 2b, is likely an off-cycle thermodynamic sink.

When analyzing the two calculated potential energy surfaces, the mechanism with de-coordination of the phosphine ligand was (for the most part) lower in energy than the more traditional pathway with de-coordination of the phosphinimine donor. Although the energy differences were not that large, typically between 1.4 to 3.5 kcal/mol, it was quite surprising that the alternative pathway was favored over the canonical Grubbs-type mechanism. The largest energy difference between the two cycles was seen between the MCB intermediates (4/4′), with a ΔΔG° value of 8.5 kcal/mol. Without being bound by any theory, it is possible that the strong π-donating ability of the phosphinimine helps stabilize alkene binding, a strong π-acidic ligand, in comparison to the phosphine, which is a strong σ-donor, but also a π-acceptor (phosphines compete with alkenes for metal-ligand back-bonding). Based on these results, it is anticipated that both cycles are energetically feasible, and likely working in parallel during the reaction. This is particularly exciting, as this is the first report of a ruthenium-based OM catalyst that generates an active species without an NHC or phosphine ligand bound.

In conjunction with DFT calculations, NMR-scale reactions with 1:20 Ru1: 1-hexene were carried out. After 5 min., when analyzing the carbene region of the 1H NMR spectrum, a small amount of starting material was present in solution, along with two major species (a doublet as well as a doublet of triplets) and one minor species (a triplet). The doublet was consistent with a methylidene complex, while the doublet of triplets was consistent with a hexylidene intermediate (both with a coordinated tricyclohexylphosphine ligand). The minor triplet, on the other hand, was tentatively assigned as a hexylidene complex with no phosphine bound, which gives some evidence for the alternative mechanism discussed above. These assignments were supported by the 34P NMR spectrum, which displayed two large phosphorus peaks at 63.7 and 57.5 ppm, corresponding to two chemically distinct triphenylphosphinimine ligands bound to ruthenium, and two large signals at 36.0 and 35.4 ppm, corresponding to coordinated tricyclohexylphosphine ligands. There were a few other minor species in solution, but no free tricyclohexylphosphine or triphenylphosphinimine could be detected. Based on these results, the two major species that were spectroscopically observed are either five- or six-coordinate ruthenium complexes with both phosphorus containing ligands bound to the metal center. This agrees with the resting states predicted by the DFT calculations (structures 2b and 6b), which were the same for either of the mechanisms that were explored. After 20 min., it was observed that the two major species decreased in intensity, whereas the minor species increased in intensity.

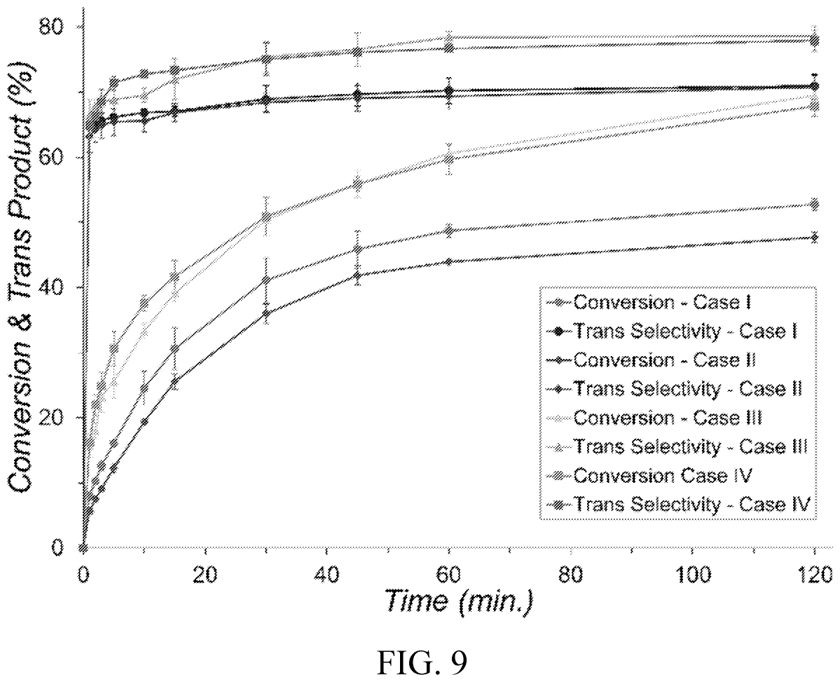

Examining Catalyst Stability. In addition to the kinetic and mechanistic experiments discussed above, 1-hexene was used as a model substrate in order to investigate the air- and moisture-sensitivity of the phosphinimine systems (FIG. 9). For Case I, the homocoupling of 1-hexene was carried out using a mixture of dried/degassed 1-hexene (2400 equiv., 10 mmol) and a solution of pre-catalyst Ru1 (1 equiv., 0.004 mmol) in 0.4 mL dried/degassed CDCl₃ (FIG. 9). When the reaction was performed, however, it was opened to air. These conditions were meant to determine the oxygen sensitivity of the active catalyst, and 71% trans selectivity for 5-decene was obtained with an overall conversion of 53% after 2 h (initial turnover frequency, TOF, of 11,500±400 h⁻¹). Next, for Case II, the reaction was carried out in air using benchtop CDCl₃ and 1-hexene (the same 1:2400 ratio of Ru1: 1-hexene was utilized, FIG. 9). A 48% conversion after 2 h was obtained, with 71% selectivity for trans-5-decene (initial TOF of 8200±500 h⁻¹). Although there was a decrease in initial TOF on going from Case I to Case II (between 20-30%), over 2 h there was not a large difference between using dried and benchtop solvents/substrates outside of a glovebox atmosphere. In contrast, for Case III the reaction was conducted inside of a glovebox using dried/degassed 1-hexene and CDCl₃ (FIG. 9). A 69% conversion after 2 h was obtained, with 79% selectivity for trans-5-decene (initial TOF of 22,600±1300 h⁻¹). This represented a substantial increase in initial TOF (approx. 100% increase), as well as higher conversion and trans selectivity over a 2 h period (in comparison to Cases I and II). Lastly, for Case IV catalysis was also carried out under a glovebox atmosphere, but degassed benchtop 1-hexene and CDCl₃ were utilized (not dried, FIG. 9). A 68% conversion after 2 h was obtained, with 78% selectivity for trans-5-decene (initial TOF of 23,300±300 h⁻¹). As such, Case III and Case IV were the same within error, suggesting that Ru1 was oxygen-sensitive, but not moisture-sensitive.

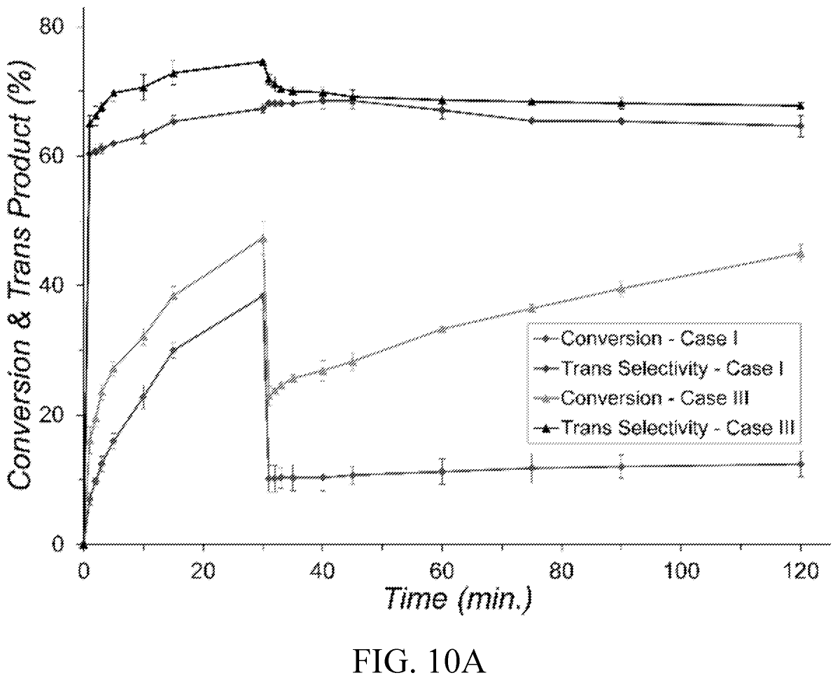

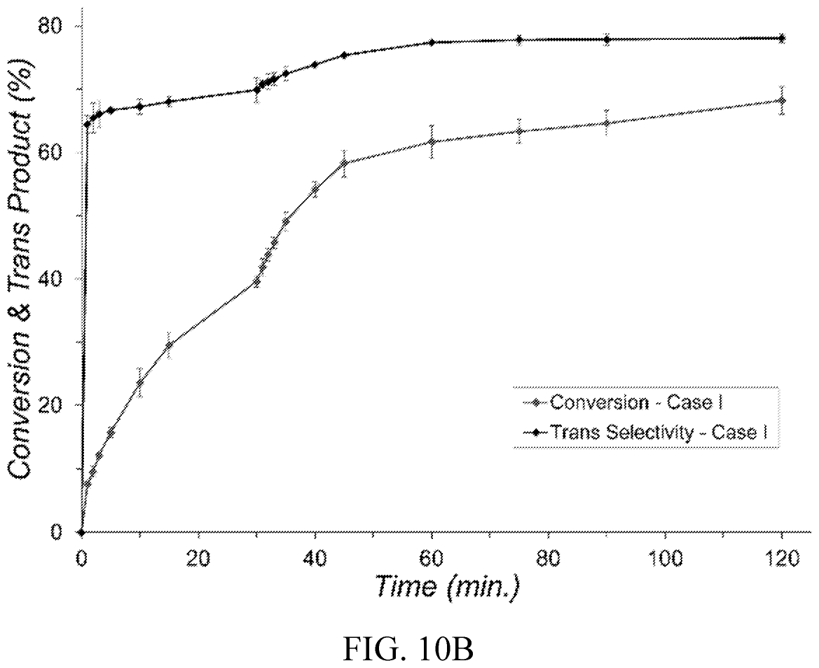

In order to further support the hypothesis that catalyst decomposition in air caused the observed decrease in activity, additional studies using conditions similar to Case I and Case III were carried out. In the first set of experiments, catalysis was allowed to proceed normally, except that after 30 min. (typically where catalytic activity dropped and conversion began to plateau) more substrate (2400 equiv.) was added (FIG. 10A). It was clear that under an inert atmosphere, the active catalyst was still present in solution and continued to convert 1-hexene to 5-decene. The reactions conducted in the presence of oxygen, in contrast, showed almost no conversion after the addition of more substrate. For the second set of experiments, catalysis was once again allowed to proceed normally, under conditions similar to Case I, except that after 30 min. more catalyst (1 equiv.) was added (FIG. 10B). This caused a rapid increase in conversion, which once again began to plateau over time (30 min. after the second equiv. of catalyst was injected, or after 60 min. total). These results gave strong evidence that catalyst decomposition in air and not substrate consumption was responsible for the observed loss of activity over time. Interestingly, the protocol with sequential addition of catalyst displayed 68% conversion after 2 h, with 78% selectivity for trans-5-decene, much like Case III and Case IV from FIG. 9.

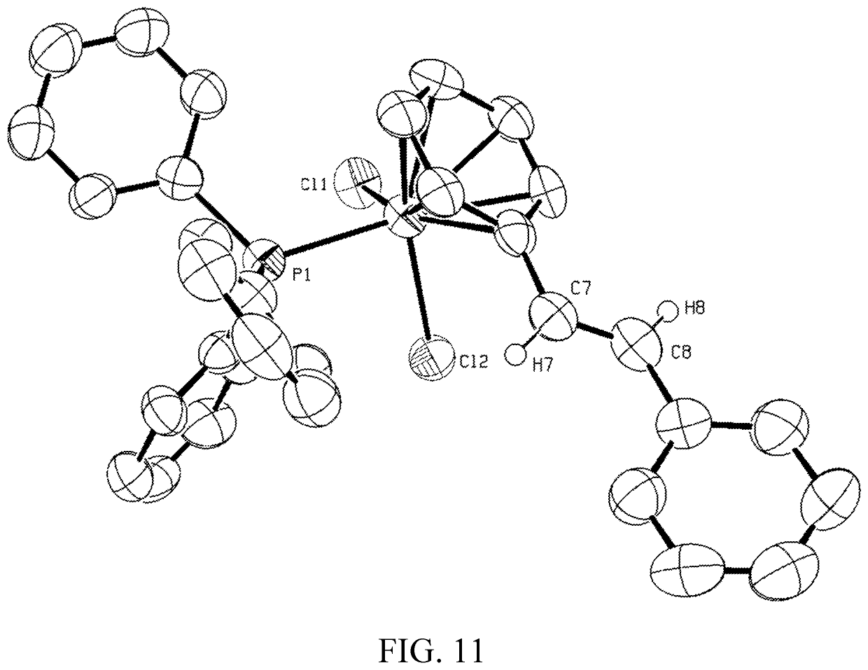

Although Ru1 was found to be relatively stable in solution at room temperature over several days, after longer periods of time, the pre-catalyst was found to slowly decompose. To probe potential decomposition pathways, a solution of Ru1 in toluene was allowed to sit for 1 week in a glovebox, and was then cooled to −30° C. After approximately one more week had elapsed, crystals suitable for single crystal X-ray diffractometry were obtained. The structure of the decomposition product, Ru2, (FIG. 11) displayed a piano stool structure with two chlorides, a triphenylphosphine ligand, and an η⁶-trans-stilbene moiety.

The structure of Ru2 was quite informative as to the potential decomposition pathways for Ru1. What immediately became apparent was that the ruthenium center had been reduced to Ru(II), and the carbene ligand, as well as the tricyclohexylphosphine moiety were missing. In addition, the triphenylphosphinimine had been reduced to a triphenylphosphine donor. A major key in rationalizing these transformations was the presence of the stilbene ligand. This gave compelling evidence for a bimolecular mechanism of decomposition, which is well documented with Schrock-type OM catalysts, but also known for ruthenium systems. With respect to the appearance of the triphenylphosphine ligand in conjunction with the disappearance of the tricyclohexylphosphine donor, it is anticipated that the NH functionality was exchanged onto the more electron-rich phosphorus atom, similar to the way phosphine oxides and phosphines can exchange oxygen atoms. In support of this hypothesis, DFT calculations showed that the formation of tricyclohexylphosphinimine was thermodynamically favorable (2.7 kcal/mol downhill, see the Supporting Information for more details). These results also gave some circumstantial support for tricyclohexylphosphine de-coordination during OM, as both bimolecular decomposition and triphenylphosphine formation would require tricyclohexylphosphine de-coordination from the metal center (to provide space at the metal and to allow nucleophilic attack of the tricyclohexylphosphine lone pair on the triphenylphosphinimine, respectively).

Substrate Scope Investigations. After completing the mechanistic studies above, the substrate scope of Ru1 with respect terminal alkenes was explored (Scheme 2 and Table 7). All reactions were carried out under a glovebox atmosphere at room temperature in CDCl₃. The homocoupling of 1-hexene was carried out using a 1:100 ratio of Ru1: substrate for comparison purposes (
Table 7, Entry 1). After 2 h, a 77.8% conversion was obtained with 83.2% trans product. After stirring the reaction mixture for an additional 22 h, 90.9% 5-decene was afforded with 81.5% trans selectivity. In order to examine the effects of steric bulk, progressively more encumbered substrates 4-methyl-1-pentene, 3-methylpent-1-ene, and 3,3-dimethyl-1-butene were examined. For the least sterically hindered substrate, 4-methyl-1-pentene, conversions of 75.3% (2 h) and 78.2% (24 h) were obtained with 74.4% and 79.1% trans selectivity, respectively (
Table 7, Entry 2). It was hypothesized that catalyst decomposition was causing the incomplete conversion after 24 h, so an additional equivalent of Ru1 was added, and the mixture was left stirring for a further 24 h. After 48 h, 86.8% conversion was obtained with 77.7% trans product (overall 1:50 ratio of Ru1: substrate). Following the same procedure, 3-methylpent-1-ene provided 34.6% overall conversion after 48 h with >99% trans selectivity (
Table 7, Entry 3), while 3,3-dimethyl-1-butene gave <1% conversion (
Table 7, Entry 4). A clear trend was observed, where catalytic efficiency dropped from Entry 1 to 4, correlating with an increase in substrate steric bulk. This phenomenon can be rationalized based on the proposed catalytic mechanism: more sterically hindered substrates are less likely to engage in an associative-type ligand substitution reaction. Additionally, substrates that are too large and require a dissociative pathway for metal binding (such as 3,3-dimethyl-1-butene) are unlikely to engage in OM with Ru1.

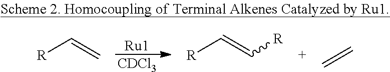

TABLE 7
Substrate Scope of Ru1 for the Homocoupling of Terminal Alkenes.^{a}

| | | Conversion (%) | | | Selectivity (% trans) | | |
|---|---|---|---|---|---|---|---|
| Entry | Substrate | 2 h | 24 h | 48 h | 2 h | 24 h | 48 h |
| 1^{b} | 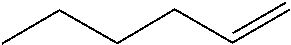 | 77.8 | 90.9 | — | 83.2 | 81.5 | — |
| 2 | 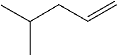 | 75.3 | 78.2 | 86.8 | 74.4 | 79.1 | 77.7 |
| 3 | 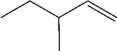 | 12.3 | 21.9 | 34.6 | >99 | >99 | >99 |
| 4 | 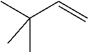 | <1 | <1 | <1 | — | — | — |
| 5 | 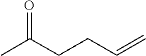 | 36.7 | 40.1 | 48.7 | 70.7 | 70.1 | 72.6 |
| 6 | 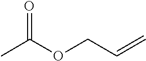 | 39.8 | 42.9 | 52.2 | 82.6 | 81.3 | 78.9 |
| 7 | 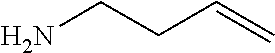 | <3 | 6.5 | 7.4 | — | 71.4 | 74.8 |
| 8^{c} | 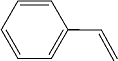 | 10.5 | 29.5 (22.1)^{d} | 28.4 (61.1)^{d} | >99 | >99 | >99 |
| 9^{c} | 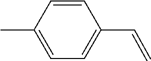 | 10.3 | 27.8 (31.9)^{d} | 20.6 (69.1)^{d} | >99 | >99 | >99 |
| 10^{c} | 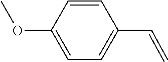 | 10.2 | 28.7 (18.5)^{d} | 21.3 (55.6)^{d} | >99 | >99 | >99 |
| 11^{c} | 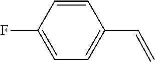 | 6.5 | 22.9 (37.4)^{d} | 18.7 (76.6)^{d} | >99 | >99 | >99 |
| 12^{c} | 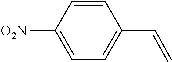 | 3.1 | 9.3 (17.5)^{d} | 8.2 (55.7)^{d} | >99 | >99 | >99 |

^{a}Reaction conditions: 0.61M substrate, 12.2 mM pre-catalyst Ru1 (two additions of Ru1) in CDCl₃ at room temperature; yields, as well as cis/trans selectivity were determined by ¹H NMR spectroscopy of the crude reaction mixtures.
^{b}6.1 mM of Ru1 was used.
^{c}0.61M 1,3,5-trimethoxybenzene was used as an internal standard.
^{d}Percentage of substrate/product consumed to generate polymerization side products.

To examine the functional group tolerance of Ru1, hex-5-en-2-one, allyl acetate, and allylamine were investigated. A similar procedure to the one used for Entries 2 to 4 in
Table 7 was employed. The presence of the carbonyl group reduced both the overall conversion, 48.7%, and trans selectivity 72.6% (
Table 7, Entry 5). Allyl acetate also demonstrated comparable results (
Table 7, Entry 6). The presence of an amine, however, caused a dramatic reduction in catalytic activity with 7.4% conversion over a 48 h period. It is uncertain at this time why Ru1 exhibited diminished catalytic ability with these substrates, but in regards to allylamine, sensitivity to protic functional groups can likely be ruled out; wet solvents could be utilized for the homocoupling of 1-hexene without a decrease in activity. It is possible that coordination of the Lewis basic groups could be responsible for the reduced catalytic efficiency, but further studies are needed.

Lastly, styrene and several of its para-substituted derivatives were tested using the protocol established for Entries 2 to 7. The more electron-rich substrates, including styrene, para-methylstyrene, and para-methoxystyrene (
Table 7, Entries 8 to 10), showed higher conversions (between 27.8 to 29.5% after 24 h) with almost perfect trans selectivity (>99%). The styrene derivatives with electron-withdrawing groups, on the other hand, para-fluorostyrene and para-nitrostyrene (
Table 7, Entries 11 and 12), became progressively less reactive the more electron poor the alkene, with conversions of 22.9 and 9.3% after 24 h, respectively (>99% trans selectivity still maintained). The overall trend seen with the styrene substrates showed good agreement with the proposed catalytic mechanism, where an associative interchange ligand substitution reaction was hypothesized to be the rate-limiting step. More electron-poor substrates would be expected to be less effective for this catalytic pathway (styrenes in general are more electron deficient than 1-hexene), which was clearly shown by para-fluorostyrene and para-nitrostyrene. In addition, it should be noted that in all cases (
Table 7, Entries 8 to 12) polymerization products formed in addition to the desired homocoupled species, which was verified using an internal standard (1,3,5-trimethoxybenzene). This likely contributed to the modest conversions seen for all of the styrene substrates. It is believed that the decomposition products of Ru1 are responsible for this observation as it has been reported that para-cymene ruthenium complexes, which are similar in structure to Ru2, are capable of affecting styrene polymerization.

### CONCLUSIONS

In summary, a ruthenium phosphinimine OM catalyst, Ru1, was synthesized, fully characterized, and its activity for the homocoupling of terminal alkenes was explored. Using 1-hexene as a model substrate, the experimentally determined rate law was found to be first-order in substrate and catalyst, indicating that both species were involved in the rate-determining step. Moreover, the empirical activation parameters ΔS^{±} and ΔH^{±} were consistent with an associative-type ligand substitution reaction. When considering the ΔG^{±}(298 K) value of Ru1, it ranked among the fastest initiating ruthenium-based OM catalysts, including the Grela nitro-substituted catalyst, the Blechert-Wakamatsu catalyst, and Grubbs 3^{rd} generation catalyst.

DFT calculations were also performed to further explore the catalytic mechanism. Two potential energy surfaces were investigated: one where the phosphinimine ligand de-coordinated (traditional mechanism) and the other where the phosphine donor de-coordinated (alternative mechanism). Although the energy differences between the two pathways were not that large, the canonical Grubbs-type mechanism was less energetically favorable than the alternative pathway. Furthermore, the theoretical calculations matched well with experimental data, and were further supported by NMR-scale reactions.

In addition to kinetic and mechanistic experiments, the stability of Ru1 was explored. Once again using 1-hexene as a model substrate, the phosphinimine OM catalyst was found to be somewhat oxygen-sensitive, but not overly moisture-sensitive. A key decomposition product of Ru1 was also crystallographically characterized, Ru2, which gave insight into the decomposition pathways of the phosphinimine catalyst. Ru2 gave strong evidence for a bimolecular mechanism of decomposition, as well as NH exchange between the tricyclohexylphosphine and triphenylphosphinimine moieties (supported by DFT calculations). Lastly, the substrate scope of Ru1 with respect to terminal olefins was explored. Two clear trends were observed: catalytic efficiency dropped with increasing steric bulk on the substrate, as well as with more electron-poor alkenes. These results were consistent with the proposed catalytic mechanism, where an associative interchange ligand substitution reaction would be hindered by sterically encumbered and/or electron-deficient olefins.

### Example II

Expanding on Example I, a second ruthenium complex was prepared according to Example I and Scheme 4 below, except L was N-heterocyclic carbene (SIMes).

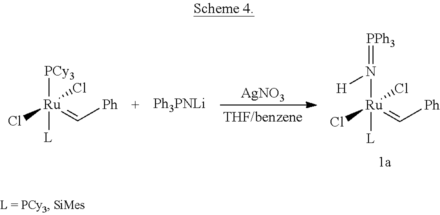

In both examples, the compounds were synthesized from Grubbs 1^{st} and 2^{nd} generation catalysts as starting materials using lithium triphenylphosphinimide (generated through the deprotonation of triphenylphosphinimine, which was synthesized according to literature procedures) and one equivalent of silver nitrate (yields between 40-48%). The NMR spectra of 1a(PCy₃) (previously described as Ru1 in EXAMPLE I but hereafter referred to as 1a(PCy₃) in EXAMPLE II) and 1a(SIMes) confirmed that the proposed phosphinimine complexes had been isolated, with a characteristic broad doublet between 4-5 ppm that corresponded to the phosphinimine N—H. Without being bound by any theory, based on literature precedent from the Fogg group, it is likely that the proton source for the phosphinimine ligand is the phenylidene donor. Therefore, during the reaction, a large portion of the ruthenium-based starting materials was thought to be decomposing. Interestingly, if either Grubbs 1^{st} or 2^{nd} generation catalyst was treated with triphenylphosphinimine itself (both in the presence and absence of silver nitrate), no reaction was observed.

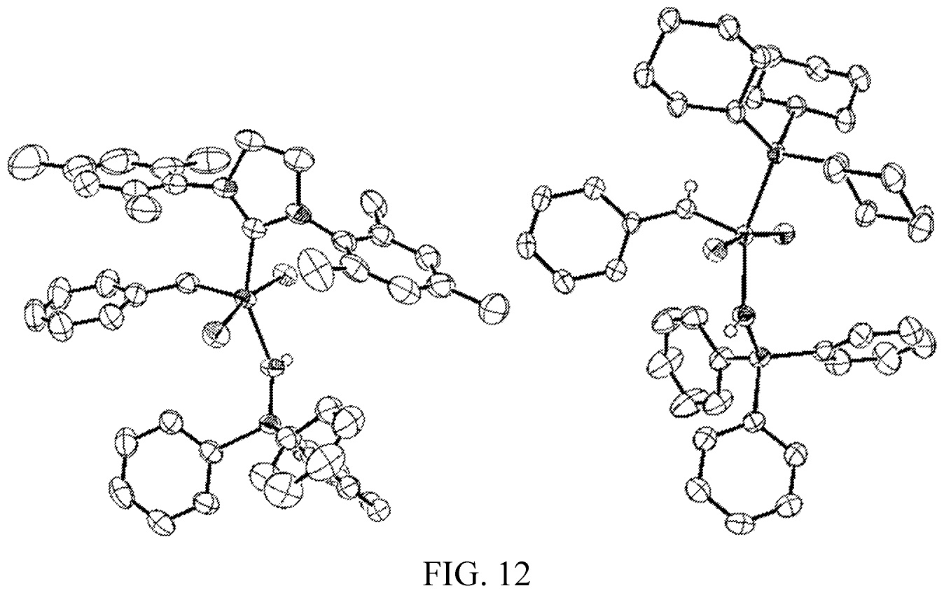

The structures of 1a(PCy₃) and 1a(SIMes) were also confirmed by single-crystal X-ray diffraction (FIG. 12). With respect to bond lengths, for 1a(PCy₃) the Ru—P distance of 2.326 Å was only slightly shortened in comparison to the ruthenium-phosphorus distances reported for Grubbs 1^{st} generation catalyst (2.38 and 2.35 Å), while the Ru—N bond length of 2.08 Å was shorter than the two known ruthenium structures with similar R₃P=N—H donors (2.121 and 2.109 Å). Similarly, for 1a(SIMes), the Ru—NHC bond length of 2.02 Å was only slightly shortened in comparison to the value reported for Grubbs 2^{nd} generation catalyst, 2.08 Å,⁸⁶ and the Ru—N bond length of 2.11 Å was still fairly short. These results were quite striking as it was initially anticipated that the tricyclohexylphosphine and NHC ligands would bind much more tightly to the metal center without a trans phosphine donor, and that they would exert a strong trans influence on the triphenylphosphinimine ligand. Based on the values obtained, however, it appears that the opposite occurred: the Ru—P and Ru—NHC bonds were not strengthened considerably, and the phosphinimine donor was tightly bound to the metal. Furthermore, these results suggest that, for these systems at least, the trans influence of the triphenylphosphinimine ligand was larger than expected a priori.

An alternative synthesis for phosphinimine ligands that does not require the heating of trimethylsilylazide in the presence of a phosphine was also developed (due to safety concerns). This straightforward and modular approach makes use an electrophilic aminating agent, O-(diphenylphosphinyl)hydroxylamine (Scheme 5).

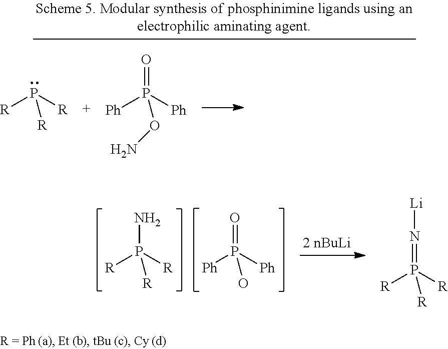

The aza phosphonium salt intermediates can be generated in very high yields (between 90-95%) and further deprotonated twice to give lithium phosphiniminde reagents. A range of phosphine starting materials were tolerated, including precursors with electron-donating (EDG) or electron-withdrawing groups (EWG), as well as species with increased steric bulk.

Initial catalytic studies investigating the OM activity of 1a(SIMes) using1-hexene as a model substrate were also carried out. While initial rates were fairly high (TOF up to 40,400 h⁻¹), overall conversions were poor (around 28.1%) due to loss of catalytic activity within the first minute of the reaction. It should be noted that the same results were obtained using wet solvent/substrate in air, as well as with dried/degassed solvent/substrate under inert atmosphere. Based on NMR studies conducted with related 1a(PCy₃), which showed rapid complex degradation under an atmosphere of ethylene, it was hypothesized that 1a(SIMes) was even more sensitive with respect to ethylene, and thus unstable under the reaction conditions. As such, it was thought that 1a(PCy₃) and 1a(SIMes) might perform better for ROMP applications, where ethylene gas would not be generated as a byproduct.

For preliminary ROMP studies, the well-established substrates norbornene and dicyclopentadiene (DCPD) were explored. Both 1a(PCy₃) and 1a(SIMes) showed exceptional activity with respect to norbornene, completing catalytic reactions within seconds. Norbornene loadings up to 48,000:1 substrate: catalyst were required to prevent 100% polymerization before a sample could be taken and quenched reproducibly (conversions of 76 and 89% in 10 s for 1a(PCy₃) and 1a(SIMes), respectively). Such high loadings, however, caused issues with respect to mass transport as the reaction medium almost immediately became gel-like. Therefore, estimates of initial TOFs are likely conservative; 4250 s-1 for 1a(SIMes) and 3800 s-1 for 1a(PCy₃). When considering selectivity, both systems generated atactic polymers, but 1a(SIMes) showed a bias towards the cis product (58% cis double bonds), whereas 1a(PCy₃) generated more of the trans product (81% trans double bonds). Further polymer characterization (polydispersity indices, etc.) is needed to determine if the phosphinimine catalysts suffer from the same chain transfer/back-biting seen for traditional Grubbs-type systems with norbornene.

In regards to DCPD, 1a(PCy₃) and 1a(SIMes) were not quite as active, but they still displayed appreciable catalytic rates with TOFs of 40 s⁻¹ and 121 s⁻¹, respectively (conversions of 26 and 76% in 15 s for 1a(PCy₃) and 1a(SIMes), respectively, with 2400:1 substrate: catalyst). More importantly, however, the phosphinimine catalysts were selective for the bicycloheptene double-bond and not the cyclopentene functionality (determined by NMR spectroscopy following established methods). Thus, linear poly (DCPD) was generated and not the extensively cross-linked product generally seen with traditional Grubbs-type catalysts (Scheme 6). It should be noted that the linear product is much more desirable than the cross-linked polymer as it displays much better ballistic resistance.

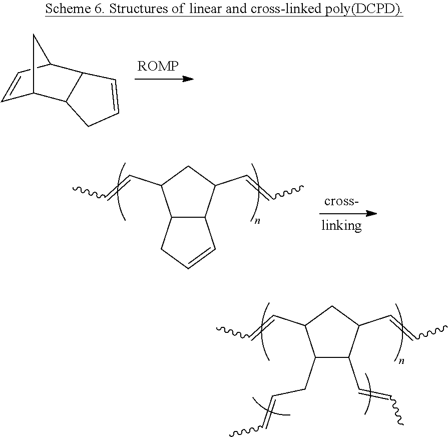

1. A method for catalyzing a metathesis reaction comprising contacting an olefin reactant with a catalyst material comprising a ruthenium phosphinimine complex.

2. The method of claim 1, wherein the ruthenium phosphinimine complex has the structure of formula (I) or (II), below: 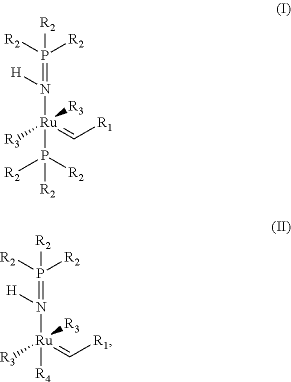
where:
each R₁ is independently selected from the group consisting of alkyls, aromatic rings, heterocyclic rings, and aromatic or heterocycle rings with substitutions;
each R₂ is independently selected from the group consisting of alkyls, aromatic rings, heterocyclic rings, and aromatic or heterocycle rings with substitutions;
each R₃ is independently selected from the group consisting of halides, cyanide, aryloxides, alkoxides, fluoroalkoxides, thiolates, pyrrolides, carboxylates, sulfates, phosphates, and nitrates; and
each R₄ is independently selected from the group consisting of heterocyclic rings and heterocycle rings with substitutions.

3. The method of claim 2, wherein the ruthenium phosphinimine complex has a Ru—N bond length of less than about 2.30 Å.

4. The method of claim 3, wherein the ruthenium phosphinimine complex has a Ru—N bond length of about 2.00 Å to about 2.25 Å.

5. The method of claim 2, wherein the ruthenium phosphinimine complex has a Ru—P bond length of not more than about 2.35 Å.

6. The method of claim 5, wherein the ruthenium phosphinimine complex has a Ru—P bond length of about 2.20 Å to about 2.35 Å.

7. The method of claim 2, wherein the ruthenium phosphinimine complex has a P—N bond length of about 1.25 Å to about 1.75 Å.

8. The method of claim 2, wherein the ruthenium phosphinimine complex has a Ru—C bond length of at least about 1.8 Å.

9. The method of claim 8, wherein the ruthenium phosphinimine complex has a Ru—C bond length of about 1.8 Å to about 2.1 Å.

10. The method of claim 1, wherein the ruthenium phosphinimine complex does not include a bound N-heterocyclic carbene and/or phosphine ligand.

11. The method of claim 1, wherein the olefin reactant comprises one or more terminal alkene molecules.

12. The method of claim 1, wherein the olefin reactant is contacted with the catalyst material by introducing the reactant into a reactor with the catalyst material, thereby converting at least a portion of the olefin reactant into one or more olefin products that are different from the olefin reactant.

13. The method of claim 12, wherein the contacting occurs at a reaction temperature of about −100° C. to about 100° C.

14. The method of claim 12, wherein after contacting the olefin reactant with the catalyst materials for at least 10 seconds, at least about 30% of the olefin reactant is converted to the one or more olefin products.

15. The method of claim 12, wherein the one or more olefin products have a trans or E selectivity of at least about 50%.

16. The method of claim 12, wherein the one or more olefin products have a cis or Z selectivity of at least about 50%.

17. A material for catalyzing an olefin metathesis reaction comprising a ruthenium phosphinimide complex.

18. The material of claim 17, wherein the ruthenium phosphinimine complex has the structure of formula (I) or (II), below: 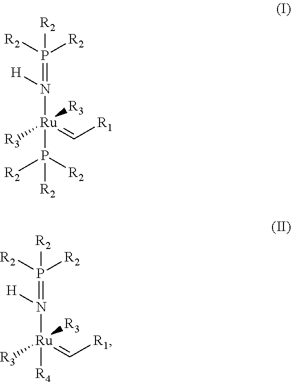
where:
each R₁ is independently selected from the group consisting of alkyls, aromatic rings, heterocyclic rings, and aromatic or heterocycle rings with substitutions;
each R₂ is independently selected from the group consisting of alkyls, aromatic rings, heterocyclic rings, and aromatic or heterocycle rings with substitutions;
each R₃ is independently selected from the group consisting of halides, cyanide, aryloxides, alkoxides, fluoroalkoxides, thiolates, pyrrolides, carboxylates, sulfates, phosphates, and nitrates; and
each R₄ is independently selected from the group consisting of heterocyclic rings and heterocycle rings with substitutions.

19. (canceled)

20. (canceled)

21. (canceled)

22. (canceled)

23. (canceled)

24. (canceled)

25. (canceled)

26. (canceled)

27. A method for synthesizing a ruthenium phosphinimine complex, the method comprising reacting a source of phosphinimine or phosphinimide ligands with a Grubbs catalyst to produce the ruthenium phosphinimine complex.

28. The method of claim 27, wherein the ruthenium phosphinimine complex has the structure of formula (I) or (II), below: 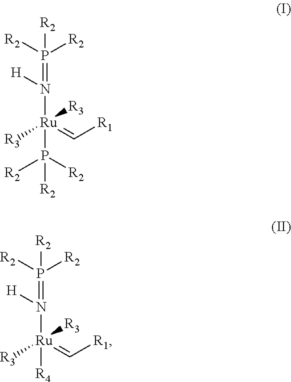
where:
each R₁ is independently selected from the group consisting of alkyls, aromatic rings, heterocyclic rings, and aromatic or heterocycle rings with substitutions;
each R₂ is independently selected from the group consisting of alkyls, aromatic rings, heterocyclic rings, and aromatic or heterocycle rings with substitutions;
each R₃ is independently selected from the group consisting of halides, cyanide, aryloxides, alkoxides, fluoroalkoxides, thiolates, pyrrolides, carboxylates, sulfates, phosphates, and nitrates; and
each R₄ is independently selected from the group consisting of heterocyclic rings and heterocycle rings with substitutions.

29. (canceled)

30. (canceled)

31. (canceled)

32. (canceled)

33. (canceled)

34. (canceled)

35. (canceled)

36. (canceled)
